(19) United States
(12) Patent Application Publication
(10) Pub. No.: US 2023/0201151 A1
(43) Pub. Date: Jun. 29, 2023

(54) FORMULATIONS AND METHODS FOR TREATING ACUTE RESPIRATORY DISTRESS SYNDROME, ASTHMA, OR ALLERGIC RHINITIS

(71) Applicants: UNIVERSITY OF FLORIDA RESEARCH FOUNDATION, INC., Gainesville, FL (US); ENTRINSIC, LLC, Norwood, MA (US)

(72) Inventors: Sadasivan VIDYASAGAR, Gainesville, FL (US); Astrid GROSCHE, Gainesville, FL (US); Xiaodong XU, Gainesville, FL (US); Damiano ANGOLI, Norwood, MA (US); Stephen J. GATTO, Norwood, MA (US)

(21) Appl. No.: 18/000,177

(22) PCT Filed: May 28, 2021
(86) PCT No.: PCT/US2021/034807

§ 371 Date: Nov. 29, 2022

Related U.S. Application Data
(60) Provisional application No. 63/136,404, filed on Jan. 12, 2021.
(60) Provisional application No. 63/088,813, filed on Oct. 7, 2020.
(60) Provisional application No. 63/080,470, filed on Sep. 18, 2020.
(60) Provisional application No. 63/032,185, filed on May 29, 2020.

(51) Int. Cl. A61K 31/198; A61K 31/405; A61P 11/06; A61P 11/02

(52) U.S. Cl. CPC A61K 31/198; A61K 31/405; A61P 11/06; A61P 11/02

(57) ABSTRACT

Formulations comprising combinations of free amino acids useful for treating ARDS, asthma, or allergic rhinitis are described herein. Use of such amino acid formulations for treating ARDS, asthma, or allergic rhinitis in a subject in need thereof; in methods for treating ARDS, asthma, or allergic rhinitis in a subject in need thereof; and/or in the preparation of a medicament for the treatment of ARDS, asthma, or allergic rhinitis are encompassed herein.

### RELATED APPLICATIONS

This application claims priority of U.S. Provisional Application No. 63/032,185 filed May 29, 2020, U.S. Provisional Application No. 63/080,470 filed Sep. 18, 2020, U.S. Provisional Application No. 63/088,813 filed Oct. 7, 2020, and U.S. Provisional Application No. 63/136,404 filed Jan. 12, 2021, the entirety of each of which is incorporated herein by reference for all purposes.

### FIELD OF THE INVENTION

Amino acid formulations, compositions, medicaments, and methods described herein are useful for treating acute respiratory distress syndrome (ARDS), asthma, or allergic rhinitis in a subject in need thereof. Subjects in need thereof may exhibit signs of respiratory distress, which signs include symptoms associated with excessive alveolar fluid. The amino acid formulations and compositions and medicaments thereof confer an increase in epithelial sodium channel (ENaC) activity, thereby reducing at least one symptom of these diseases. ARDS is a syndrome associated with a variety of diseases, including coronavirus disease 2019 (COVID-19). Use of amino acid formulations described herein for treating ARDS, asthma, or allergic rhinitis in a subject in need thereof and in the preparation of a medicament for the treatment of ARDS, asthma, or allergic rhinitis, as well as methods for treating ARDS, asthma, or allergic rhinitis are encompassed herein.

### BACKGROUND OF THE INVENTION

SARS-CoV-2, which causes coronavirus disease 2019 (COVID-19), predominantly infects airway and alveolar epithelial cells, vascular endothelial cells, and macrophages. SARS-CoV-2 infection frequently leads to fatal inflammatory responses and acute respiratory distress syndrome (ARDS), which is associated with high mortality in COVID-19 patients. ARDS develops in 42% of patients presenting with COVID-19 pneumonia, and 61-81% of those are admitted to an intensive care unit (ICU). In ˜20% of COVID-19 patients, the disease is severe and such patients need oxygen therapy or mechanical ventilation. COVID-19 ARDS patients have a median time of 8.5 days on a ventilator after symptom onset and typically, such patients have poor prognoses following such supportive therapy. ARDS causes diffuse alveolar damage in the lung. Intriguingly, COVID-19 ARDS patients have worse outcomes than ARDS patients due to other causes. Despite advancement in treatment protocols, patients with ARDS continue to experience high mortality rates.

### SUMMARY

Covered embodiments are defined by the claims, not this summary. This summary is a high-level overview of various aspects and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings, and each claim.

ENaC and barrier function play a key role in alveolar fluid clearance and their disruption contributes to ARDS as seen in COVID-19. Poor recognition of SARS-CoV-2 by innate immune mechanisms leads to early activation of Th1 and Th2 responses and suppression of Treg cell responses. This altered immune response results in the classic cytokine storm, which ultimately leads to disruption of ENaC activity and barrier function. Prior to the present results, little was known about the timeline and quantity of cytokines involved in disruption of ENaC activity and barrier function. This lack of understanding has contributed to a paucity of treatment options to address ARDS.

Based on electrophysiological and immunofluorescence techniques presented herein, the present inventors demonstrate that ENaC activity decreased earlier than barrier disruption and Th2 cytokines (IL-4 and IL-13) contributed more significantly to these inhibitory effects than cytokines from innate (IFN-γ), Th1 (TNF-α) and Treg (TGF-β) immune responses.

As described herein, primary normal human bronchial epithelial cells (HBECs) were exposed to representative cytokines, and combinations thereof that are released during COVID-19 in a dose- and time-dependent evaluation. To explore the potential that an amino acid formulation could be used to treat ARDS, at least in part by increasing ENaC function, the present inventors evaluated a plurality of amino acid formulations, including one designated AA-EC01, for their ability to modulate ENaC activity in a model system of primary HBECs exposed to selected cytokines characteristic of the COVID-19 immune response. As described herein, AA-EC01 is an exemplary amino acid formulation that improved ENaC function and decreased MUC5AC expression in HBECs when exposed to IL-13 at a dose and incubation time that showed maximum ENaC inhibition. AA-EC01 also increased ENaC expression and decreased IL-6 secretion within periciliary membranes of HBECs incubated with a cytokine cocktail. Accordingly, results presented herein demonstrate the beneficial effect of AA-EC01 on ENaC function in an in vitro model system of the ARDS-associated inflammatory response. By virtue of its ability to recover ENaC activity, AA-EC01 has the potential to be the first therapeutic formulation designed to improve the outcome of patients with ARDS following SARS-CoV-2 or other pulmonary virus infections. AA-EC01 can be used as a stand-alone therapeutic agent or may be used in a combinatorial therapeutic approach with other therapeutic agents currently used to treat patients with ARDS.

AA-EC01 is also presented as a therapeutic agent for treating asthma. For treating asthma, AA-EC01 may be used as a stand-alone therapeutic agent or may be used in a combinatorial therapeutic approach with other therapeutic agents currently used to treat patients with asthma.

AA-EC01 is also presented as a therapeutic agent for treating allergic rhinitis. For treating allergic rhinitis, AA-EC01 may be used as a stand-alone therapeutic agent or may be used in a combinatorial therapeutic approach with other therapeutic agents currently used to treat patients with allergic rhinitis.

In some embodiments, a pharmaceutical formulation for use in treating ARDS, asthma, or allergic rhinitis in a subject in need thereof is presented, wherein the formulation comprises a therapeutically effective combination of free amino acids: the free amino acids consisting essentially of or consisting of a therapeutically effective amount of free amino acids of arginine and lysine; and a therapeutically effective amount of at least one of free amino acids of glutamine, tryptophan, tyrosine, cysteine, asparagine, or threonine, or any combination thereof, wherein the therapeutically effective combination of free amino acids is formulated for delivery to the lungs for treating ARDS or asthma and the therapeutically effective combination of free amino acids is sufficient to reduce fluid accumulation in the lungs of the subject; or wherein the therapeutically effective combination of free amino acids is formulated for delivery to the nasal passages for treating allergic rhinitis and the therapeutically effective combination of free amino acids is sufficient to reduce fluid accumulation in the nasal passages of the subject; and optionally, at least one pharmaceutically acceptable carrier, buffer, electrolyte, adjuvant, excipient, or water, or any combination thereof.

In some embodiments of the pharmaceutical formulation, the free amino acids consist essentially of or consist of a therapeutically effective amount of free amino acids of arginine and lysine; and a therapeutically effective amount of at least one of free amino acids of glutamine, tryptophan, tyrosine, cysteine, or asparagine, or any combination thereof.

In some embodiments of the pharmaceutical formulation, the free amino acids consist essentially of or consist of a therapeutically effective amount of free amino acids of arginine, lysine, and glutamine; and a therapeutically effective amount of at least one of free amino acids of tryptophan, tyrosine, cysteine, asparagine, or threonine, or any combination thereof.

In some embodiments of the pharmaceutical formulation, the free amino acids consist essentially of or consist of a therapeutically effective amount of free amino acids of arginine, lysine, and glutamine; and a therapeutically effective amount of at least one of free amino acids of tryptophan, tyrosine, cysteine, or asparagine, or any combination thereof.

In some embodiments of the pharmaceutical formulation, the pharmaceutical formulation is sterile.

In some embodiments of the pharmaceutical formulation, a concentration of each of the free amino acids present in the pharmaceutical formulation ranges from 0.1 mM to 30 mM or 0.5 mM to 30 mM. In some embodiments, a concentration of each of the free amino acids present in the pharmaceutical formulation ranges from 0.1 mM to 15 mM or 0.5 mM to 15 mM. In some embodiments, a concentration of each of the free amino acids present in the pharmaceutical formulation ranges from 0.1 mM to 10 mM or 0.5 mM to 10 mM.

In some embodiments of the pharmaceutical formulation, the pH of the pharmaceutical formulation ranges from 2.5 to 8.0, 3.0 to 8.0, 3.5 to 8.0, 4.0 to 8.0, 4.5 to 8.0, 4.5 to 6.5, 5.5 to 6.5, 5.0 to 8.0, 5.5 to 8.0, 6.0 to 8.0, 6.5 to 8.0, 7.0 to 8.0, or 7.5 to 8.0.

In some embodiments of the pharmaceutical formulation, the concentration of arginine ranges from 4 mM to 10 mM; the concentration of arginine ranges from 6 mM to 10 mM; the concentration of arginine ranges from 7 mM to 9 mM; the concentration of arginine ranges from 7.2 mM to 8.8 mM; or the concentration of arginine is 8 mM; the concentration of lysine ranges from 4 mM to 10 mM; the concentration of lysine ranges from 6 mM to 10 mM; the concentration of lysine ranges from 7 mM to 9 mM; the concentration of lysine ranges from 7.2 mM to 8.8 mM; or the concentration of lysine is 8 mM; the concentration of glutamine ranges from 4 mM to 10 mM; the concentration of glutamine ranges from 6 mM to 10 mM; the concentration of glutamine ranges from 7 mM to 9 mM; the concentration of glutamine ranges from 7.2 mM to 8.8 mM; or the concentration of lysine is 8 mM; the concentration of tryptophan ranges from 4 mM to 10 mM; the concentration of tryptophan ranges from 6 mM to 10 mM; the concentration of tryptophan ranges from 7 mM to 9 mM; the concentration of tryptophan ranges from 7.2 mM to 8.8 mM; or the concentration of tryptophan is 8 mM; the concentration of tyrosine ranges from 0.1 mM to 1.2 mM; the concentration of tyrosine ranges from 0.4 mM to 1.2 mM; the concentration of tyrosine ranges from 0.6 mM to 1.2 mM; the concentration of tyrosine ranges from 0.8 mM to 1.2 mM; or the concentration of tyrosine is 1.2 mM; the concentration of cysteine ranges from 4 mM to 10 mM; the concentration of cysteine ranges from 6 mM to 10 mM; the concentration of cysteine ranges from 7 mM to 9 mM; the concentration of cysteine ranges from 7.2 mM to 8.8 mM; or the concentration of cysteine is 8 mM; the concentration of asparagine ranges from 4 mM to 10 mM; the concentration of asparagine ranges from 6 mM to 10 mM; the concentration of asparagine ranges from 7 mM to 9 mM; the concentration of asparagine ranges from 7.2 mM to 8.8 mM; or the concentration of asparagine is 8 mM; the concentration of threonine ranges from 4 mM to 10 mM; the concentration of threonine ranges from 6 mM to 10 mM; the concentration of threonine ranges from 7 mM to 9 mM; the concentration of threonine ranges from 7.2 mM to 8.8 mM; or the concentration of threonine is 8 mM; or any combination thereof.

In some embodiments of the pharmaceutical formulation, the therapeutically effective combination of free amino acids consists essentially of or consists of a therapeutically effective amount of free amino acids of arginine, lysine, tryptophan, tyrosine, and glutamine, and optionally, asparagine. In some embodiments of the pharmaceutical formulation, the therapeutically effective combination of free amino acids consists essentially of or consists of a therapeutically effective amount of free amino acids of arginine, lysine, tryptophan, tyrosine, and glutamine. In some embodiments of the pharmaceutical formulation, arginine is present at a concentration ranging from 6 mM to 10 mM, lysine is present at a concentration ranging from 6 mM to 10 mM, tryptophan is present at a concentration ranging from 6 mM to 10 mM, tyrosine is present at a concentration ranging from 0.1 mM to 1.2 mM, and glutamine is present at a concentration ranging from 6 mM to 10 mM. In some embodiments of the pharmaceutical formulation, arginine is present at a concentration ranging from 7.2 mM to 8.8 mM, lysine is present at a concentration ranging from 7.2 mM to 8.8 mM, tryptophan is present at a concentration ranging from 7.2 mM to 8.8 mM, tyrosine is present at a concentration ranging from 0.8 mM to 1.2 mM, and glutamine is present at a concentration ranging from 7.2 mM to 8.8 mM. In some embodiments of the pharmaceutical formulation, arginine is present at a concentration of 8 mM, lysine is present at a concentration of 8 mM, tryptophan is present at a concentration of 8 mM, tyrosine is present at a concentration of 1.2 mM, and glutamine is present at a concentration of 8 mM.

In some embodiments of the pharmaceutical formulation, the therapeutically effective combination of free amino acids consists essentially of or consists of a therapeutically effective amount of free amino acids of arginine, lysine, tryptophan, and glutamine, and optionally, asparagine. In some embodiments of the pharmaceutical formulation, the therapeutically effective combination of free amino acids consists essentially of or consists of a therapeutically effective amount of free amino acids of arginine, lysine, tryptophan, and glutamine. In some embodiments of the pharmaceutical formulation, arginine is present at a concentration ranging from 6 mM to 10 mM, lysine is present at a concentration ranging from 6 mM to 10 mM, tryptophan is present at a concentration ranging from 6 mM to 10 mM, and glutamine is present at a concentration ranging from 6 mM to 10 mM. In some embodiments of the pharmaceutical formulation, arginine is present at a concentration ranging from 7.2 mM to 8.8 mM, lysine is present at a concentration ranging from 7.2 mM to 8.8 mM, tryptophan is present at a concentration ranging from 7.2 mM to 8.8 mM, and glutamine is present at a concentration ranging from 7.2 mM to 8.8 mM. In some embodiments of the pharmaceutical formulation, arginine is present at a concentration of 8 mM, lysine is present at a concentration of 8 mM, tryptophan is present at a concentration of 8 mM, and glutamine is present at a concentration of 8 mM.

In some embodiments of the pharmaceutical formulation, the therapeutically effective combination of free amino acids consists essentially of or consists of a therapeutically effective amount of free amino acids of arginine, lysine, tyrosine, and glutamine, and optionally, asparagine. In some embodiments of the pharmaceutical formulation, the therapeutically effective combination of free amino acids consists essentially of or consists of a therapeutically effective amount of free amino acids of arginine, lysine, tyrosine, and glutamine. In some embodiments of the pharmaceutical formulation, arginine is present at a concentration ranging from 6 mM to 10 mM, lysine is present at a concentration ranging from 6 mM to 10 mM, tyrosine is present at a concentration ranging from 0.1 mM to 1.2 mM, and glutamine is present at a concentration ranging from 6 mM to 10 mM. In some embodiments of the pharmaceutical formulation, arginine is present at a concentration ranging from 7.2 mM to 8.8 mM, lysine is present at a concentration ranging from 7.2 mM to 8.8 mM, tyrosine is present at a concentration ranging from 0.8 mM to 1.2 mM, and glutamine is present at a concentration ranging from 7.2 mM to 8.8 mM. In some embodiments of the pharmaceutical formulation, arginine is present at a concentration of 8 mM, lysine is present at a concentration of 8 mM, tyrosine is present at a concentration of 1.2 mM, and glutamine is present at a concentration of 8 mM.

In some embodiments of the pharmaceutical formulation, the therapeutically effective combination of free amino acids consists essentially of or consists of a therapeutically effective amount of free amino acids of arginine, lysine, glutamine, cysteine, and asparagine. In some embodiments of the pharmaceutical formulation, arginine is present at a concentration ranging from 6 mM to 10 mM, lysine is present at a concentration ranging from 6 mM to 10 mM, glutamine is present at a concentration ranging from 6 mM to 10 mM, cysteine is present at a concentration ranging from 6 mM to 10 mM, and asparagine is present at a concentration ranging from 6 mM to 10 mM. In some embodiments of the pharmaceutical formulation, arginine is present at a concentration ranging from 7.2 mM to 8.8 mM, lysine is present at a concentration ranging from 7.2 mM to 8.8 mM, glutamine is present at a concentration ranging from 7.2 mM to 8.8 mM, cysteine is present at a concentration ranging from 7.2 mM to 8.8 mM, and asparagine is present at a concentration ranging from 7.2 mM to 8.8 mM. In some embodiments of the pharmaceutical formulation, arginine is present at a concentration of 8 mM, lysine is present at a concentration of 8 mM, glutamine is present at a concentration of 8 mM, cysteine is present at a concentration of 8 mM, and asparagine is present at a concentration of 8 mM.

In some embodiments of the pharmaceutical formulation, the therapeutically effective combination of free amino acids consists essentially of or consists of a therapeutically effective amount of free amino acids of arginine, lysine, and tryptophan, and optionally, asparagine. In some embodiments of the pharmaceutical formulation, the therapeutically effective combination of free amino acids consists essentially of or consists of a therapeutically effective amount of free amino acids of arginine, lysine, and tryptophan. In some embodiments of the pharmaceutical formulation, arginine is present at a concentration ranging from 6 mM to 10 mM, lysine is present at a concentration ranging from 6 mM to 10 mM, and tryptophan is present at a concentration ranging from 6 mM to 10 mM. In some embodiments of the pharmaceutical formulation, arginine is present at a concentration ranging from 7.2 mM to 8.8 mM, lysine is present at a concentration ranging from 7.2 mM to 8.8 mM, and tryptophan is present at a concentration ranging from 7.2 mM to 8.8. In some embodiments of the pharmaceutical formulation, arginine is present at a concentration of 8 mM, lysine is present at a concentration of 8 mM, and tryptophan is present at a concentration of 8 mM.

In some embodiments of the pharmaceutical formulation, the therapeutically effective combination of free amino acids consists essentially of or consists of a therapeutically effective amount of free amino acids of arginine, lysine, tryptophan, threonine, and tyrosine, and optionally, asparagine. In some embodiments of the pharmaceutical formulation, the therapeutically effective combination of free amino acids consists essentially of or consists of a therapeutically effective amount of free amino acids of arginine, lysine, tryptophan, threonine, and tyrosine. In some embodiments of the pharmaceutical formulation, arginine is present at a concentration ranging from 6 mM to 10 mM, lysine is present at a concentration ranging from 6 mM to 10 mM, tryptophan is present at a concentration ranging from 6 mM to 10 mM, threonine is present at a concentration ranging from 6 mM to 10 mM, and tyrosine is present at a concentration ranging from 0.1 mM to 1.2 mM. In some embodiments of the pharmaceutical formulation, arginine is present at a concentration ranging from 7.2 mM to 8.8 mM, lysine is present at a concentration ranging from 7.2 mM to 8.8 mM, tryptophan is present at a concentration ranging from 7.2 mM to 8.8 mM, threonine is present at a concentration ranging from 7.2 mM to 8.8 mM, and tyrosine is present at a concentration ranging from 0.8 mM to 1.2 mM. In some embodiments of the pharmaceutical formulation, arginine is present at a concentration of 8 mM, lysine is present at a concentration of 8 mM, tryptophan is present at a concentration of 8 mM, threonine is present at a concentration of 8 mM, and tyrosine is present at a concentration of 1.2 mM.

In some embodiments of the pharmaceutical formulation, the therapeutically effective combination of free amino acids consists essentially of or consists of a therapeutically effective amount of free amino acids of arginine, lysine, tryptophan, threonine, and glutamine, and optionally, asparagine. In some embodiments of the pharmaceutical formulation, the therapeutically effective combination of free amino acids consists essentially of or consists of a therapeutically effective amount of free amino acids of arginine, lysine, tryptophan, threonine, and glutamine. In some embodiments of the pharmaceutical formulation, arginine is present at a concentration ranging from 6 mM to 10 mM, lysine is present at a concentration ranging from 6 mM to 10 mM, tryptophan is present at a concentration ranging from 6 mM to 10 mM, threonine is present at a concentration ranging from 6 mM to 10 mM, and glutamine is present at a concentration ranging from 6 mM to 10 mM. In some embodiments of the pharmaceutical formulation, arginine is present at a concentration ranging from 7.2 mM to 8.8 mM, lysine is present at a concentration ranging from 7.2 mM to 8.8 mM, tryptophan is present at a concentration ranging from 7.2 mM to 8.8 mM, threonine is present at a concentration ranging from 7.2 mM to 8.8 mM, and glutamine is present at a concentration ranging from 7.2 mM to 8.8 mM. In some embodiments of the pharmaceutical formulation, arginine is present at a concentration of 8 mM, lysine is present at a concentration of 8 mM, tryptophan is present at a concentration of 8 mM, threonine is present at a concentration of 8 mM, and glutamine is present at a concentration of 8 mM.

In some embodiments of the pharmaceutical formulation, the therapeutically effective combination of free amino acids consists essentially of or consists of a therapeutically effective amount of free amino acids of arginine, lysine, tryptophan, tyrosine, glutamine, and threonine, and optionally, asparagine. In some embodiments of the pharmaceutical formulation, the therapeutically effective combination of free amino acids consists essentially of or consists of a therapeutically effective amount of free amino acids of arginine, lysine, tryptophan, tyrosine, glutamine, and threonine. In some embodiments of the pharmaceutical formulation, arginine is present at a concentration ranging from 6 mM to 10 mM, lysine is present at a concentration ranging from 6 mM to 10 mM, tryptophan is present at a concentration ranging from 6 mM to 10 mM, tyrosine is present at a concentration ranging from 0.1 mM to 1.2 mM, glutamine is present at a concentration ranging from 6 mM to 10 mM, and threonine is present at a concentration ranging from 6 mM to 10 mM. In some embodiments of the pharmaceutical formulation, arginine is present at a concentration ranging from 7.2 mM to 8.8 mM, lysine is present at a concentration ranging from 7.2 mM to 8.8 mM, tryptophan is present at a concentration ranging from 7.2 mM to 8.8 mM, tyrosine is present at a concentration ranging from 0.8 mM to 1.2 mM, glutamine is present at a concentration ranging from 7.2 mM to 8.8 mM, and threonine is present at a concentration ranging from 7.2 mM to 8.8 mM. In some embodiments of the pharmaceutical formulation, arginine is present at a concentration of 8 mM, lysine is present at a concentration of 8 mM, tryptophan is present at a concentration of 8 mM, tyrosine is present at a concentration of 1.2 mM, glutamine is present at a concentration of 8 mM, and threonine is present at a concentration of 8 mM.

In some embodiments, the pharmaceutical formulation further comprises at least one pharmaceutically acceptable carrier, buffer, electrolyte, adjuvant, excipient, or water, or any combination thereof.

In some embodiments of the pharmaceutical formulation, at least one of the free amino acids or each of the free amino acids comprises L-amino acids. In some embodiments of the pharmaceutical formulation, all of the amino acids are L-amino acids.

In some embodiments of the pharmaceutical formulation, the pharmaceutical formulation is formulated for administration by a pulmonary, inhalation, or intranasal route. In some embodiments of the pharmaceutical formulation, the pharmaceutical formulation is formulated for administration via inhalation or nasal administration.

In some embodiments of the pharmaceutical formulation, the subject is a mammal. In some embodiments of the pharmaceutical formulation, the mammal is a human, cat, dog, pig, horse, cow, sheep, or goat. In some embodiments of the pharmaceutical formulation, the mammal is a human. In some embodiments of the pharmaceutical formulation, the human is a baby.

In some embodiments of the pharmaceutical formulation, the subject is afflicted with coronavirus disease 2019 (COVID-19).

In some embodiments of the pharmaceutical formulation, the pharmaceutical formulation reduces excessive fluid accumulation in the lungs of the subject afflicted with ARDS or asthma, thereby reducing at least one symptom associated with ARDS or asthma. In some embodiments of the pharmaceutical formulation, the pharmaceutical formulation reduces excessive fluid accumulation in the nasal passages of the subject afflicted with allergic rhinitis, thereby reducing at least one symptom associated with allergic rhinitis. Reduction in excessive fluid accumulation is due, in part, to an increase in ENaC activity.

In some embodiments of the pharmaceutical formulation, the pharmaceutical formulation is for use in treating ARDS, asthma, or allergic rhinitis. In some embodiments thereof, the pharmaceutical formulation is administrable via at least one of a pulmonary, inhalation, or intranasal route. In some embodiments thereof, the pharmaceutical formulation is administrable via inhalation or nasal administration.

In some embodiments of the pharmaceutical formulation, the pharmaceutical formulation is for use in the manufacture of a medicament for treating ARDS, asthma, or allergic rhinitis. In some embodiments thereof, the medicament is administrable via at least one of a pulmonary, inhalation, or intranasal route. In some embodiments thereof, the medicament is administrable via inhalation or nasal administration.

In some embodiments of the pharmaceutical formulation, the pharmaceutical formulation is used in a method for treating ARDS, asthma, or allergic rhinitis in a subject in need thereof, the method comprising: administering to the subject in need thereof at least one of the pharmaceutical formulations described herein, wherein the administering reduces fluid accumulation in the lung, thereby reducing at least one symptom associated with ARDS or asthma in the subject, or the administering reduces fluid accumulation in the nasal passages of the subject, thereby reducing at least one symptom associated with allergic rhinitis in the subject.

In some embodiments of the method, the pharmaceutical formulation is administered via a pulmonary, inhalation, or intranasal route. In some embodiments of the method, the pharmaceutical formulation is administered via inhalation or nasal administration.

In some embodiments of the pharmaceutical formulation, a pharmaceutical formulation comprising a combination of free amino acids is presented: the free amino acids consisting essentially of or consisting of a therapeutically effective amount of free amino acids of arginine and lysine; and a therapeutically effective amount of at least one of free amino acids of glutamine, tryptophan, tyrosine, cysteine, asparagine, or threonine, or any combination thereof, and optionally, at least one pharmaceutically acceptable carrier, buffer, electrolyte, adjuvant, excipient, or water, or any combination thereof.

In some embodiments of the pharmaceutical formulation, a pharmaceutical formulation comprising a therapeutically effective combination of free amino acids is presented: the free amino acids consisting essentially of or consisting of a therapeutically effective amount of free amino acids of arginine and lysine; and a therapeutically effective amount of at least one of free amino acids of glutamine, tryptophan, tyrosine, cysteine, or asparagine, or any combination thereof.

In some embodiments of the pharmaceutical formulation, a pharmaceutical formulation comprising a combination of free amino acids is presented: the free amino acids consisting essentially of or consisting of a therapeutically effective amount of free amino acids of arginine, lysine, and glutamine; and a therapeutically effective amount of at least one of free amino acids of tryptophan, tyrosine, cysteine, asparagine, or threonine, or any combination thereof.

In some embodiments of the pharmaceutical formulation, the therapeutically effective combination of free amino acids consists essentially of or consists of a therapeutically effective amount of free amino acids of arginine, lysine, tryptophan, tyrosine, and glutamine.

In some embodiments of the pharmaceutical formulation, the therapeutically effective combination of free amino acids consists essentially of or consists of a therapeutically effective amount of free amino acids of arginine, lysine, glutamine, cysteine, and asparagine.

In some embodiments of the pharmaceutical formulation, the therapeutically effective combination of free amino acids consists essentially of or consists of a therapeutically effective amount of free amino acids of arginine, lysine, tryptophan, and glutamine.

In some embodiments of the pharmaceutical formulation, the therapeutically effective combination of free amino acids consists essentially of or consists of a therapeutically effective amount of free amino acids of arginine, lysine, tyrosine, and glutamine.

In some embodiments of the pharmaceutical formulation, a device comprising a pharmaceutical formulation described herein or a medicament comprising a pharmaceutical formulation described herein is presented, wherein the device is configured to deliver the pharmaceutical formulation or the medicament to the lungs or nasal passages of the subject in need thereof. Exemplary such devices include: inhalers, nebulizers, nasal spray containers, and nasal drop containers.

All combinations of separately described embodiments are envisaged.

### BRIEF DESCRIPTION OF THE DRAWINGS

Some embodiments of the disclosure are herein described, by way of example only, with reference to the accompanying drawings. With specific reference now to the drawings in detail, it is stressed that the embodiments shown are by way of example and for purposes of illustrative discussion of embodiments of the disclosure. In this regard, the description taken with the drawings makes apparent to those skilled in the art how embodiments of the disclosure may be practiced.

FIG. 1: Schematic representation of the pathogenesis of SARS-CoV-2 infection through alveolus and the surrounding microcapillary bed, inhibiting sodium channel ENaC in the process.

FIG. 2: ENaC current in human bronchial epithelial cells in the presence of different concentrations of IL-13. N=6 tissues.

FIG. 3: Time required for IL-12 to result in maximum reduction in ENaC current N=6 tissues.

FIG. 4: Time required for IL-13 to result in maximum reduction in ENaC Current. N=6 tissues

FIGS. 5A and 5B: HBEC cells grown on permeable inserts and treated with IL-13 for 4 days and 14 days. FIG. 5A. HBEC showing increased ENaC current in the presence of the formulation AAF01 (also referred to herein as AA-EC01) when compared to Ringer solution. FIG. 5B. Bumetanide-sensitive anion current decreased in the presence of the AAF01 when compared to HBEC in Ringer solution. N=6 tissues.

FIGS. 6A and 6B: AAF01 decreased chloride secretion in IL-13 treated HBEC. FIG. 6A. Jnet Basal WT54 and WT59; FIG. 6B. Jnet After Bumetanide WT54 and WT59. AAF01 decreases IL-13 induced Cl secretion back to normal (Day 0).

FIG. 7A-D: Effect of select amino acid formulations on benzamil-sensitive currents (ENaC activity) and bumetanide-sensitive currents (anion current) in fully differentiated primary HBEC treated with 20 ng of IL-13 for 4 and 14 days. Mean±SEM; ANOVA with * P<0.05 when compared to Ringer control (n=3).

FIGS. 8A and 8B: Effect of select amino acid formulations on benzamil-sensitive currents (ENaC activity) and bumetanide-sensitive currents (anion current) in primary HBEC when treated with 20 ng of IL-13 for 4 and 14 days. Mean±SEM; ANOVA with P<0.05 (n=3).

FIG. 9: ENaC Activity in Human Bronchial Epithelial Cells after Exposure to Increasing Concentrations of TNF-α for 7 Days. Human bronchial epithelial cells (HBEC) were treated with different concentrations of TNF-α (0.00005, 0.0005, 0.005, 0.05, 0.5, 5, 50 or 500 ng/mL media) for 7 days.

FIG. 10: ENaC Activity in Human Bronchial Epithelial Cells after Exposure to Increasing Concentrations of IFN-γ for 7 Days. HBEC were treated with IFN-γ (0.00005, 0.0005, 0.005, 0.05, 0.5, 5, 50 or 500 ng/mL media) for 7 days.

FIG. 11: ENaC Activity in Human Bronchial Epithelial Cells after Exposure to Increasing Concentrations of TGF-β1 for 7 Days. HBEC were treated with TGF-β1 (0.00005, 0.0005, 0.005, 0.05, 0.5, 5, 50 or 500 ng/mL media) for 7 days.

FIG. 12: Effect of select amino acid formulations on ENaC Activity in Human Bronchial Epithelial Cells after Exposure to TNF-α, IFN-γ and TGF-β1 for 7 Days. HBEC were treated with TNF-α (1.2 ng/mL media), IFN-γ (0.875 ng/mL media), and TGF-β1 (2.6 ng/mL) for 7 days. Naive cells: Age-matched normal healthy cells. Select “5AA formulation” (8 mM arginine, 8 mM lysine, 8 mM cysteine, 8 mM asparagine, 8 mM glutamine); NC (8 mM aspartic acid, 8 mM threonine, 8 mM leucine).

FIG. 13A-13D: Dose- and time-dependent effect of IFN-γ on benzamil-sensitive I_{sc} and TEER in HBECs. (13A) Dose-dependent effect of IFN-γ on benzamil-sensitive I_{sc} was analyzed after incubation of HBECs with increasing concentrations of IFN-γ (5×10⁻⁵ to 500 ng/mL) for 7 days. Delta I_{sc} was calculated from I_{sc} before and 15 minutes after adding 6 μM benzamil apically to the ringer solution in Ussing chambers. (13B) Dose-dependent effect of IFN-γ on TEER was analyzed in after incubation of HBECs with increasing concentrations of IFN-γ (5×10⁻⁵ to 500 ng/mL) for 7 days. TEER was recorded after 30 minutes while bathing in ringer solution in Ussing chambers. (13C) Time-dependent effect of IFN-γ on benzamil-sensitive I_{sc} was analyzed after incubation of HBECs with 1 ng/mL IFN-γ for 16 days, and data were analyzed on day 2, 4, 6, 8, 10, 12, 14, and 16. Delta I_{sc} was calculated from I_{sc} before and 15 minutes after adding 6 μM benzamil apically to the ringer solution in Ussing chambers. (13D) Time-dependent effect of IFN-γ on TEER was analyzed after incubation of HBECs with 1 ng/mL IFN-γ for 16 days, and data were analyzed on day 2, 4, 6, 8, 10, 12, 14, and 16. TEER was recorded after 30 minutes while bathing in ringer solution in Ussing chambers. All values are normalized to controls (0 ng/mL cytokine/day 0), and data are presented as means±SEM (n=2 donors with N=2 independent experiments per group). Statistical significance was tested with Mann-Whitney test for pairwise comparison with control (* P<0.05).

FIG. 14A-14D: Dose- and time-dependent effect of TNF-α on benzamil-sensitive I_{sc} and TEER in HBECs. (14A) Dose-dependent effect of TNF-α on benzamil-sensitive I_{sc} was analyzed after incubation of HBECs with increasing concentrations of TNF-α (5×10⁻⁵ to 500 ng/mL) for 7 days. Delta I_{sc} was calculated from I_{sc} before and 15 minutes after adding 6 μM benzamil apically to the ringer solution in Ussing chambers. (14B) Dose-dependent effect of TNF-α on TEER was analyzed after incubation of HBECs with increasing concentrations of TNF-α (5×10⁻⁵ to 500 ng/mL) for 7 days. TEER was recorded after 30 minutes while bathing in ringer solution in Ussing chambers. (14C) Time-dependent effect of TNF-α on benzamil-sensitive I_{sc} was analyzed after incubation of HBECs with 1 ng/mL TNF-α for 16 days, and data were analyzed on day 2, 4, 6, 8, 10, 12, 14, and 16. Delta I_{sc} was calculated from I_{sc} before and 15 minutes after adding 6 μM benzamil apically to the ringer solution in Ussing chambers. (14D) Time-dependent effect of TNF-α on TEER was analyzed after incubation of HBECs with 1 ng/mL TNF-α for 16 days, and data were analyzed on day 2, 4, 6, 8, 10, 12, 14, and 16. TEER was recorded after 30 minutes while bathing in ringer solution in Ussing chambers. All values are normalized to controls (0 ng/mL cytokine/day 0), and data are presented as means±SEM (n=2 donors with N=2 independent experiments per group). Statistical significance was tested with Mann-Whitney test for pairwise comparison with control (* P<0.05).

FIG. 15A-15D: Dose-dependent effect of an IFN-γ and TNF-α cocktail, and time-dependent effect of IL-4 on benzamil-sensitive I_{sc} and TEER in HBECs. (15A) Dose-dependent effect of an IFN-γ and TNF-α cocktail on benzamil-sensitive I_{sc} was analyzed after incubation of HBECs with IFN-γ and TNF-α at 0.05, 0.5, 2.5, 5 or 10 ng/mL each for 7 days. Delta I_{sc} was calculated from I_{sc} before and 15 minutes after adding 6 μM benzamil apically to the ringer solution in Ussing chambers. (15B) Dose-dependent effect of an IFN-γ and TNF-α cocktail on TEER was analyzed after incubation of HBECs with IFN-γ and TNF-α at 0.05, 0.5, 2.5, 5 or 10 ng/mL each for 7 days. TEER was recorded after 30 minutes while bathing in ringer solution in Ussing chambers. (15C) Time-dependent effect of IL-4 on benzamil-sensitive I_{sc} was analyzed after incubation of HBECs with 2 ng/mL IL-4 for 14 days, and data were analyzed on day 2, 4, 6, 8, 10, 12, and 14. Delta I_{sc} was calculated from I_{sc} before and 15 minutes after adding 6 μM benzamil apically to the ringer solution in Ussing chambers. (15D) Time-dependent effect of IL-4 on TEER was analyzed after incubation of HBECs with 2 ng/mL IL-4 for 14 days, and data were analyzed on day 2, 4, 6, 8, 10, 12, and 14. TEER was recorded after 30 minutes while bathing in ringer solution in Ussing chambers. All values are normalized to controls (0 ng/mL cytokine/day 0), and data are presented as means±SEM (n=2 donors with N=2 independent experiments per group). Statistical significance was tested with Mann-Whitney test for pairwise comparison with control (* P<0.05).

FIG. 16A-16D: Dose- and time-dependent effect of IL-13 on benzamil-sensitive I_{sc} and TEER in HBECs. (16A) Dose-dependent effect of IL-13 on benzamil-sensitive I_{sc} was analyzed after incubation of HBECs with increasing concentrations of IL-13 (0.1 to 64 ng/mL) for 14 days. Delta I_{sc} was calculated from I_{sc} before and 15 minutes after adding 6 μM benzamil apically to the ringer solution in Ussing chambers. (16B) Dose-dependent effect of IL-13 on TEER was analyzed after incubation of HBECs with increasing concentrations of IL-13 (0.1 to 64 ng/mL) for 14 days. TEER was recorded after 30 minutes while bathing in ringer solution in Ussing chambers. (16C) Time-dependent effect of IL-13 on benzamil-sensitive I_{sc} was analyzed after incubation of HBECs with 20 ng/mL IL-13 for 16 days, and data were analyzed on day 2, 4, 6, 8, 10, 12, 14, and 16. Delta I_{sc} was calculated from I_{sc} before and 15 minutes after adding 6 μM benzamil apically to the ringer solution in Ussing chambers. (16D) Time-dependent effect of IL-13 on TEER was analyzed after incubation of HBECs with 20 ng/mL IL-13 for 16 days, and data were analyzed on day 2, 4, 6, 8, 10, 12, 14, and 16. TEER was recorded after 30 minutes while bathing in ringer solution in Ussing chambers. All values are normalized to controls (0 ng/mL cytokine/day 0), and data are presented as means±SEM (n=2 donors with N=2 independent experiments per group). Statistical significance was tested with Mann-Whitney test for pairwise comparison with control (* P<0.05).

FIG. 17A-17D: Dose- and time-dependent effect of TGF-β1 on benzamil-sensitive I_{sc} and TEER in HBECs. (17A) Dose-dependent effect of TGF-β1 on benzamil-sensitive I_{sc} was analyzed after incubation of HBECs with increasing concentrations of TGF-β1 (5×10⁻⁵ to 50 ng/mL) for 7 days. Delta I_{sc} was calculated from I_{sc} before and 15 minutes after adding 6 μM benzamil apically to the ringer solution in Ussing chambers. (17B) Dose-dependent effect of TGF-β1 on TEER was analyzed after incubation of HBECs with increasing concentrations of TGF-β1 (5×10⁻⁵ to 50 ng/mL) for 7 days. TEER was recorded after 30 minutes while bathing in ringer solution in Ussing chambers. (17C) Time-dependent effect of TGF-β1 on benzamil-sensitive I_{sc} was analyzed after incubation of HBECs with 1 ng/mL TGF-β1 for 16 days, and data were analyzed on day 2, 4, 6, 8, 10, 12, 14, and 16. Delta I_{sc} was calculated from I_{sc} before and 15 minutes after adding 6 μM benzamil apically to the ringer solution in Ussing chambers. (17D) Time-dependent effect of TGF-β1 on TEER was analyzed after incubation of HBECs with 1 ng/mL TGF-β1 for 16 days, and data were analyzed on day 2, 4, 6, 8, 10, 12, 14, and 16. TEER was recorded after 30 minutes while bathing in ringer solution in Ussing chambers. All values are normalized to controls (0 ng/mL cytokine/day 0), and data are presented as means±SEM (n=2 donors with N=2 independent experiments per group). Statistical significance was tested with Mann-Whitney test for pairwise comparison with control (* P<0.05).

FIG. 18A-18B: Effect of AA-EC01 on benzamil-sensitive I_{sc} and TEER in HBECs, and schematic illustration of AA-EC01 affecting ENaC and immune response in COVID-19-associated ARDS. (18A) Effect of AA-EC01 on benzamil-sensitive I_{sc} was analyzed after incubation of HBECs with 20 ng/mL IL-13 for 14 days. Delta I_{sc} was calculated from I_{sc} before and 15 minutes after adding 6 μM benzamil apically to ringer solution, AA-EC01 or AANC (negative control) in Ussing chambers. (18B) Effect of AA-EC01 on TEER was analyzed after incubation of HBECs with 20 ng/mL IL-13 for 14 days. TEER was recorded after 30 minutes while bathing in ringer solution, AA-EC01 or AANC (negative control) in Ussing chambers. All values are normalized to control (0 ng/mL IL-13), and data are presented as means±SEM (n=2 donors with N=2 independent experiments per group). After significance was confirmed between the groups with Kruskal-Wallis, Mann-Whitney test was used for pairwise comparison (* P<0.05).

### DETAILED DESCRIPTION

Among those benefits and improvements that have been disclosed, other objects and advantages of this disclosure will become apparent from the following description taken in conjunction with the accompanying figures. Detailed embodiments of the present disclosure are disclosed herein; however, it is to be understood that the disclosed embodiments are merely illustrative of the disclosure that may be embodied in various forms. In addition, each of the examples given regarding the various embodiments of the disclosure which are intended to be illustrative, and not restrictive.

ARDS is associated with high mortality in COVID-19. ARDS is characterized by a cytokine storm with impaired alveolar liquid clearance (ALC), alveolar-capillary hyperpermeability and vascular and epithelial leakage, leading to leakage of protein-rich fluid from pulmonary capillaries into the interstitial and alveolar space, causing pulmonary edema. Under normal conditions, the airways facilitate gas exchange across the alveolar lumen and the capillary network embedded in inter-alveolar septa. ENaC mediates electrogenic sodium absorption, followed by passive water absorption and maintains an optimum moisture content for mucociliary clearance. ENaC is, however, inhibited at multiple stages of COVID-19 pathogenesis, which leads to accumulation of fluid in the alveoli. Oxygen supplementation and ventilator support enhances inflammation, triggering superoxide, peroxynitrite formation and Nitric Oxide Synthase (NOS) uncoupling, and damaging barrier and transport proteins, including ENaC.

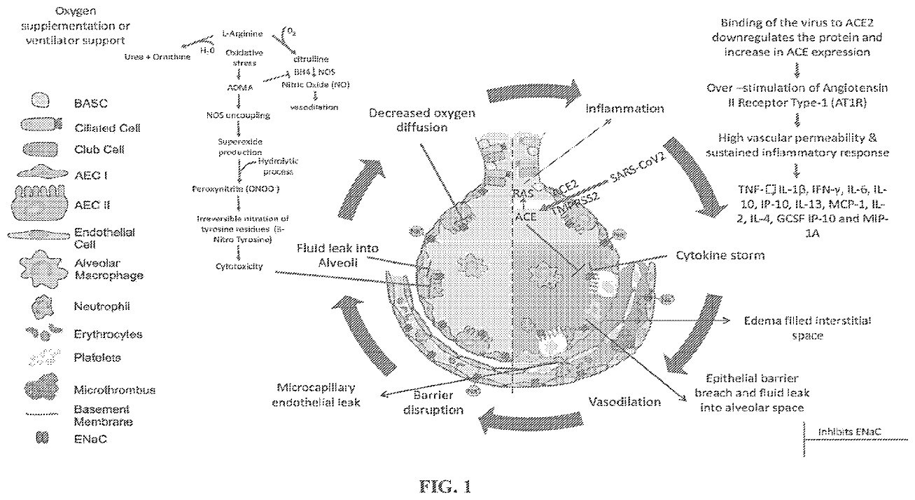

The above cascade of events is depicted schematically in FIG. 1. SARS-CoV-2 inhibition of ENaC activity occurs at the following stages: 1) Transmembrane protease serine Si member 2 (TMPRSS2), a host cell factor essential for proteolytic activation of the virus, and consequently COVID-19 spread and pathogenesis; 2) Angiotensin Converting Enzyme 2 (ACE2) that upregulates Angiotensin Converting Enzyme (ACE) and Renin Angiotensin System (RAS); 3) Cytokine storm secondary to ACE and RAS activation leads to elevated levels of TNF-α, IL-1β, IFN-γ, IL-6, IL-10, IP-10, IL-13, MCP-1, IL-2, IL-4, GCSF IP-10 and MIP-1A; 4) Breakdown of the epithelial and endothelial barrier, leading to fluid leak into the alveoli, thereby reducing gas exchange; and 5) Uncoupling of NOS secondary to inflammation and local oxygen increase within the alveoli.

The only available treatments for ARDS are supplemental oxygen and use of a ventilator to help dissolve more oxygen through the edema fluid-filled alveolar spaces and to increase available oxygen at the blood-air-barrier. Oxygen supplementation and ventilator support, however, enhance inflammation and favor eNOS uncoupling, superoxide formation, increased peroxynitrite (ONOO⁻), and irreversible nitration of cysteine residues of various cellular proteins, including membrane associated proteins like ENaC in the epithelium and the surrounding vasculature. Damage to ENaC and other cellular proteins that contribute to essential cellular functions such as, for example, transport and intracellular and intercellular structural integrity creates further damage that adversely impacts lung tissue integrity.

The high mortality in COVID-19 patients receiving supplemental oxygen therapy and mechanical ventilation may be associated with the above-outlined cascade of insults. Indeed, mortality in these patients ranges from 65% to 94%, which statistics have prompted debate as to the merit of using ventilators for SARS-CoV-2 patients. It is, moreover, noteworthy that subjects suffering from COVID-19-mediated ARDS have far worse outcomes than those afflicted with ARDS due to other causes.

The present inventors have developed assays to investigate potential therapeutic regimen for addressing ARDS and have developed model systems in which to address the challenges of treating ARDS, particularly ARDS in COVID-19 patients/subjects. Accordingly, the model systems described herein were designed to address the significant clinical problems associated with ARDS, whether associated with COVID-19 or independent of COVID-19, and present solutions to such clinical problems by way of providing amino acid formulations such as those described herein. Turning first to the in vitro model systems used to address these clinical problems, the present inventors used differentiated primary human bronchial epithelial cells (HBEC) exposed to various inflammatory promoting agents to recapitulate features of ARDS.

In some embodiments of the model system, the present inventors showed that exposure of differentiated HBEC to IL-13 leads to inhibition of ENaC and impairment of barrier function. Accordingly, the present inventors developed an experimental system based on this finding wherein these features of ARDS were recapitulated to an extent comparable to that observed in the lung of afflicted subjects/patients.

The experimental system developed comprising differentiated HBEC exposed to IL-13 described herein was used as a model system for evaluating the effect of various amino acid formulations on increasing ENaC activity and improving barrier function. Using this model system, a plurality of amino acid formulations were identified and characterized based on their ability to increase ENaC transport protein activity, as measured by their ability to increase ENaC current, and to improve barrier function. See Tables 1 and 2 below. An exemplary such formulation is the five amino acid formulation (AAF01). As shown herein, AAF01 increased ENaC current, decreased anion current, and improved barrier function in HBEC treated with IL-13 for 14 days. AAF01 was selected at least in part due to its ability to reduce chloride secretion and improve barrier function.

These findings provide evidence that AAF01 and other exemplary amino acid formulations described herein may be used to treat subjects afflicted with COVID-19, particularly those subjects exhibiting at least one symptom of ARDS. AAF01 and other exemplary amino acid formulations described herein may also be used to treat subjects afflicted with asthma or allergic rhinitis, conditions in which Th2 cytokines (e.g., IL-4 and IL-13) play significant roles. Based on the results presented herein, AAF01 and other exemplary amino acid formulations described herein may act at least in part via their ability to increase ENaC activity and improve alveolar fluid clearance.

Results presented herein demonstrate that AAF01:
Increased amiloride/benzamil-sensitive ENaC current
Increased ENaC protein levels
Increased NHE3 protein levels (ENaC independent sodium absorption)
Increased tight junction protein levels and function
AAF01 can be used for treating ARDS associated with COVID and other forms of pneumonia, as well as asthma and allergic rhinitis.
AAF01 can be delivered via a variety of means, including without limitation: in an aerosolized form such as that delivered by a nebulizer, inhaler, or nasal atomizer.
AAF01 be used in combination with other agents used for treating SARS-CoV-2, asthma, and/or allergic rhinitis.

Based on results presented herein, AAF01, AAF03, and AAF07 were selected as exemplary formulations for treating ARDS, at least in part because each of the formulations confers increases in ENaC activity in model systems described herein that recapitulate features of respiratory distress. Each of AAF01, AAF03, and AAF07 were selected as exemplary formulations due to their ability to reduce chloride secretion and/or reduce barrier permeability in model systems described herein that recapitulate features of respiratory distress, such as those observed in ARDS or asthma, which features include excess alveolar fluid accumulation. The ability to reduce chloride secretion and/or reduce barrier permeability also conferred upon each of AAF01, AAF03, and AAF07 the ability to serve as therapeutic formulations for treating allergic rhinitis by reducing excessive fluid accumulation in nasal passages of a subject in need thereof.

TABLE 1

| AAF07 | AAF01* | AAF03 |
|---|---|---|
| Lysine | Lysine | Lysine |
| Arginine | Tryptophan | Tryptophan |
| Tyrosine | Arginine | Arginine |
| Glutamine | Tyrosine | Glutamine |
| | Glutamine | |

*AAF01 (also referred to herein as AA-EC01)

TABLE 2

| AAF02 | AAF04 | AAF05 | AAF06 |
|---|---|---|---|
| Lysine | Lysine | Lysine | Lysine |
| Tryptophan | Tryptophan | Tryptophan | Tryptophan |
| Arginine | Arginine | Arginine | Arginine |
| | Threonine | Threonine | Threonine |
| | Tyrosine | Glutamine | Tyrosine |
| | | | Glutamine |

Exemplary amino acid formulations described herein [e.g., AAF01, AAF03, AAF07, and the select 5AA formulation (arginine, lysine, cysteine, asparagine, and glutamine)] are useful for treating ARDS, asthma, or allergic rhinitis in a subject in need thereof. ARDS or asthma may be associated with alveolar fluid accumulation and therefore, symptomatic relief can be conferred by improving alveolar fluid clearance. The exemplary amino acid formulations described herein improve alveolar fluid clearance, at least in part by upregulating ENaC function, as reflected by increased sodium and fluid absorption. Accordingly, the amino acid formulations described herein are presented for use in treating ARDS or asthma, wherein improving alveolar fluid clearance is desired. The amino acid formulations described herein for use in treating ARDS or asthma may be used alone or in combination with at least one other active pharmaceutical ingredient (API) used to treat each of these disorders. The property of being able to improve alveolar fluid clearance also underscores the utility of exemplary amino acid formulations described herein in the preparation of a medicament for treating ARDS or asthma, wherein such medicaments improve alveolar fluid clearance and thus, confer symptomatic relief to subjects afflicted with these disorders. The amino acid formulations described herein may be the only API in the medicament or may be present in combination with at least one other API used to treat ARDS or asthma. Exemplary amino acid formulations described herein may also be used in methods for treating subjects in need thereof who have ARDS or asthma, which are associated with alveolar fluid accumulation. Methods for treating ARDS or asthma may call for administering the amino acid formulations described herein alone or in combination with at least one other API used to treat ARDS or asthma.

Exemplary amino acid formulations described herein (e.g., AAF01, AAF03, AAF07, and the select 5AA formulation) are useful for treating allergic rhinitis in a subject in need thereof. Allergic rhinitis is associated with excessive fluid in the nasal passages and therefore, symptomatic relief can be conferred by improving fluid clearance from the nasal passages. The exemplary amino acid formulations described herein improve fluid clearance from the sinuses and/or nasal passages, at least in part by upregulating ENaC function, as reflected by increased sodium and fluid absorption. Accordingly, the amino acid formulations described herein are presented for use in treating allergic rhinitis. The amino acid formulations described herein for use in treating allergic rhinitis may be used alone or in combination with at least one other API used to treat allergic rhinitis. The property of being able to improve fluid clearance from the nasal passages also underscores the utility of exemplary amino acid formulations described herein in the preparation of a medicament for treating allergic rhinitis, wherein reducing excessive nasal secretions is desired. The amino acid formulations described herein may be the only API in the medicament or may be present in combination with at least one other API used to treat allergic rhinitis. Exemplary amino acid formulations described herein may also be used in methods for treating subjects in need thereof who have allergic rhinitis. Methods for treating allergic rhinitis may call for administering the amino acid formulations described herein alone or in combination with at least one other API used to treat allergic rhinitis.

In some embodiments, a concentration of each of the free amino acids present in the formulation ranges from 0.1 mM to 30 mM or 0.5 mM to 30 mM. In some embodiments, a concentration of each of the free amino acids present in a formulation ranges from 0.1 mM to 15 mM or 0.5 mM to 15 mM. In some embodiments, a concentration of each of the free amino acids present in the formulation ranges from 0.1 mM to 10 mM or 0.5 mM to 10 mM. In some embodiments, a concentration of each of the free amino acids present in the formulation ranges from 4 mM to 12 mM, from 5 mM to 12 mM, from 6 mM to 12 mM, from 4 mM to 10 mM, from 5 mM to 10 mM, from 6 mM to 10 mM, from 4 mM to 9 mM, from 5 mM to 9 mM, or from 6 mM to 9 mM, with the exception of tyrosine, which ranges from 0.1-1.2 mM, from 0.5-1.2 mM, from 0.6-1.2 mM, or from 0.8-1.2 mM (e.g., about 1.2 mM). In some embodiments, a concentration of each of the free amino acids present in the formulation ranges from 7 mM to 9 mM (e.g., about 8 mM), with the exception of tyrosine, which ranges from 0.8-1.2 mM (e.g., about 1.2 mM). In some embodiments, the formulation is AAF01 (also referred to herein as AA-EC01) as follows: 8 mM lysine, 8 mM tryptophan, 8 mM arginine, 8 mM glutamine, and 1.2 mM tyrosine.

In some embodiments, the pH of a formulation described herein ranges from 2.5 to 8.0, 3.0 to 8.0, 3.5 to 8.0, 4.0 to 8.0, 4.5 to 8.0, 4.5 to 6.5, 5.5 to 6.5, 5.0 to 8.0, 5.5 to 8.0, 6.0 to 8.0, 6.5 to 8.0, 7.0 to 8.0, or 7.5 to 8.0.

In some embodiments wherein the formulations are delivered via nebulizer (inhalation or solution suspensions), the pH of the formulation may range between a pH of 4.5 to 6.5, which reduces the tendency of subjects to sneeze responsive to administration.

In some embodiments wherein the formulations are delivered via nasal spray or nasal atomizer, the pH of the formulation may range between a pH of 4.5 to 6.5. In some embodiments, the pH of the formulation may range between a pH of 5.5 to 6.5. Commercially available nasal spray products typically have pHs in the range of 3.5 to 7.0. The pH of the nasal epithelium typically ranges from 5.5-6.5. The average baseline human nasal pH is about 6.3.

In some embodiments, the dose per spray puff (left and right nostril): potency <5 mg/dose; volume maximally 100 μl/spray puff: solubility >50 mg/ml; drug in solution: pH approximately 5.5, osmolality 290-500 mosm/kg.

In some embodiments, the formulations described herein are delivered via nasal irrigation in, e.g., a suitable saline solution. Suitable saline solutions are commercially available or alternatively, can be made at home. A suitable saline solution may comprise 1-2 cups of warm water (e.g., distilled, sterile, or boiled) in which ¼ to ½ teaspoon of non-iodized salt and a pinch of baking soda are dissolved.

Application Device: The intended use and the pharmaceutical form of a formulation intended for nasal administration (e.g., lavages, drops, squirt systems, sprays) dictate the application devices that may be used. The dose (volume per puff normally only 100 μl), the dosing options (single vs. multiple), the subject (consumer, healthcare professional, patient, child, elderly individual) and a subject's state of health also influence the choice of the application device. Transmucosal nasal delivery and absorption benefits from the avoidance of gastrointestinal destruction and hepatic first-pass metabolism.

In some embodiments, the formulations described herein are used sequentially to address the phase of the immune response to a pathogen (e.g., SARS-CoV-2). Accordingly, an amino acid formulation suitable for treating early phase disease is replaced by an amino acid formulation suitable for treating late phase disease as disease progresses from early to late phase. In some embodiments, a formulation that counteracts the pathological consequences of cytokines characteristic of innate immunity (e.g., IFN-γ) and/or Th1 cellular response (e.g., TNF-α) is administered in early phases of an immune response to a pathogen or condition (e.g., chronic or acute). Exemplary formulations for counteracting pathological consequences of cytokines characteristic of innate immunity and/or Th1 cellular response include a first formulation: wherein such a first formulation comprises a therapeutically effective combination of free amino acids consisting essentially of a therapeutically effective amount of arginine and lysine; and a therapeutically effective amount of at least one of a free amino acid of cysteine, asparagine, or glutamine, or any combination thereof. Such immune responses are observed in the early immune response to respiratory conditions caused by pathogens, such as those mounted in response to SARS-CoV-2. As the immune response to, e.g., SARS-CoV-2, progresses over time, the cytokine expression panel can change to that characteristic of a Th2 cell response (e.g., IL-4 and IL-13). Once the immune response has begun to progress to a Th2 cell response, a second formulation comprising exemplary amino acid formulations such as, e.g., AAF01, AAF03, or AAF07 may be used to replace the first formulation. Evidence presented herein, demonstrates that, e.g., AAF01 (also referred to herein as AA-EC01) is therapeutically suited to address the pathological consequences of Th2 type cytokines by at least partially restoring ENaC activity.

Based on results presented herein, a therapeutic regimen may comprise a first amino acid formulation that counteracts the pathological effects of cytokines characteristic of innate immunity and/or Th1 cells, at least in part by restoring ENaC activity, followed by a second amino acid formulation that counteracts the pathological effects of cytokines characteristic of Th2 cells, at least in part by restoring ENaC activity. First and second amino acid formulations are administrable or may be administered sequentially and separately or sequentially with overlapping dosing, with a gradual tapering off of the amount of the first amino acid formulation as increasing amounts of the second amino acid formulation are added, until only the second amino acid formulation is administered. The timing for administration of the first and second amino acid formulations may be determined by an attending physician, based on clinical signs and presentation of symptoms.

In some embodiments, a subject may be assessed to determine if the subject exhibits an immune response in which the predominant immune response comprises production of cytokines characteristic of innate immunity and/or Th1 cells, or production of cytokines characteristic of Th2 cells, or exhibits an immune response in which the initial immune response comprises production of cytokines characteristic of innate immunity and/or Th1 cells and is later followed by an immune response comprising production of cytokines characteristic of Th2 cells. Such an assessment may be used to tailor the amino acid formulation to the subject's genetics, condition, environment, and lifestyle, thereby facilitating precision medicine.

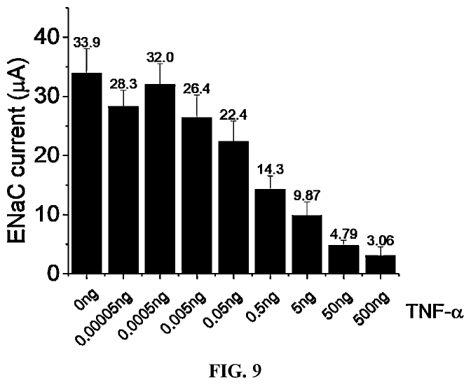

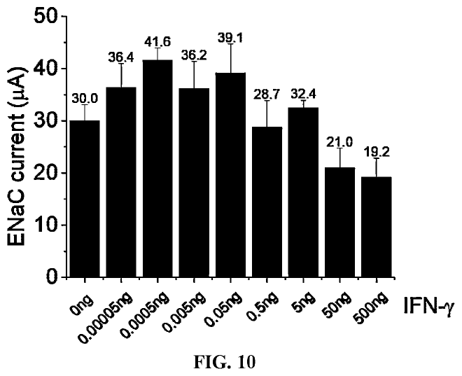

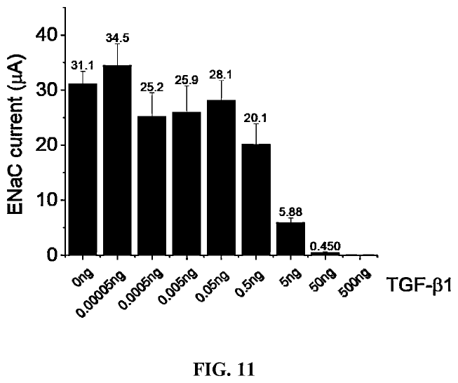

Further to the above, the effect of cytokine-induced inflammation on ENaC activity and barrier function was explored as detailed in the Examples and drawings presented herein. As described herein, ENaC is critical in the maintenance of the epithelial fluid layer. Some cytokines, such as TNF-α, TGF-β, IFN-γ, and IL-6 at high concentrations are strongly associated with lung injury and ARDS, and as shown herein, decrease ENaC activity and function, thus preventing fluid clearance from the airways in COVID-19 patients. To explore effects of these cytokines in disease etiology and progression, the present inventors exposed normal human bronchial epithelial cells to a cocktail of three cytokines (TNF-α, TGF-β1, IFN-γ) for 7 days to analyze their effect on ENaC activity and subsequently selected amino acid formulations that reverse the adverse effects of increased cytokine levels on ENaC function. See FIGS. 9-12. FIG. 9, for example, shows that ENaC current decreased with increasing concentrations of TNF-α. FIG. 10, for example, shows that ENaC current increased when cells were treated with lower concentrations of IFN-γ (0.00005 to 0.05 ng/mL media). ENaC current returned to baseline (untreated) levels when exposed to higher levels of IFN-γ, but then decreased relative to baseline when cells were treated with higher concentrations of IFN-γ (>0.05 ng/mL media). FIG. 11, for example, shows that ENaC current decreased with increasing concentrations of TGF-β1.

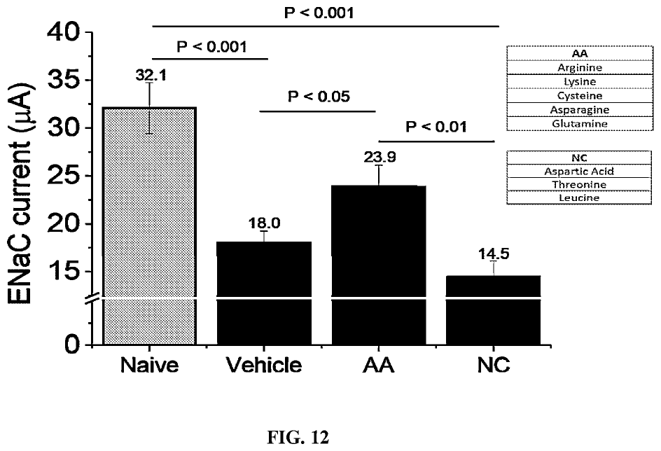

FIG. 12, for example, shows that exposure of HBEC to TNF-α, IFN-γ, and TGF-β1 (cytokine cocktail) for 7 days significantly decreased ENaC activity (vehicle) as compared to HBEC not exposed to the cytokine cocktail (naive). The term “vehicle” as used in FIG. 12 refers to the solution into which AAs were introduced to generate the 5AA formulation and the NC formulation and thus, serves as a negative control for the AA formulations. As shown in FIG. 12, the select 5AA formulation (AA; arginine, lysine, cysteine, asparagine, and glutamine) conferred significant recovery of ENaC activity in HBEC exposed to TNF-α, IFN-γ, and TGF-β1 as compared to naive cells. In some embodiments, the select 5AA formulation comprises 8 mM arginine, 8 mM lysine, 8 mM cysteine, 8 mM asparagine, and 8 mM glutamine conferred significant recovery of ENaC activity in HBEC exposed to TNF-α, IFN-γ, and TGF-β1 as compared to naive cells. The NC formulation (aspartic acid, threonine, and leucine) did not improve the cytokine-induced reduction of ENaC activity. Indeed, the NC formulation decreased ENaC activity further in HBEC that were exposed to the cytokine cocktail relative to HBEC exposed to the cytokine cocktail and vehicle.

As detailed herein above, ARDS is a common respiratory manifestation of coronavirus disease-19 (COVID-19) and other viral lung infections. ARDS results from impaired alveolar fluid clearance (AFC) which causes pulmonary edema, poor ventilation and reduced oxygen saturation. Under normal circumstances, airway surface liquid (ASL) composed of a thin layer of periciliary fluid (˜7 μm) and mucus contributes to 600 mL of fluid spanning ˜75 m² surface area and facilitates mucociliary function to clear dust and other foreign particles from the airways. A complex interplay of apical anion channel activity and reabsorption by ENaC creates an osmotic gradient for passive water movement and maintains AFC. Reduced ENaC function, as seen for example in influenza virus infection, causes decreased AFC that persists beyond active viral replication. Barrier disruption triggers exudation of protein-rich fluid from pulmonary microvascular capillaries into the alveoli resulting in noncardiogenic pulmonary edema and hyaline membrane formation that severely impairs AFC.

ENaC and barrier function are affected at multiple stages of COVID-19 pathogenesis. The type II transmembrane serine proteases (TMPRSS2), disintegrin and metallopeptidase domain 17 (ADAM17) that contribute to the ability of SARS-CoV-2 to bind angiotensin-converting enzyme 2 (ACE2) and enter the host cell also inhibit ENaC function. See FIG. 1. Binding of SARS-CoV-2 to ACE2 results in decreased ACE2 levels causing an imbalance between the renin-angiotensin-aldosterone system (RAAS) and tissue kallikrein-kinin system (KKS) with elevated angiotensin II (Ang II) and kinins. Ang II and kinins inhibit ENaC function both directly and through release of pro-inflammatory cytokines including TNF-α and IL-6. In SARS-CoV-2 infection, virus-associated molecular patterns are poorly recognized by pattern recognition receptors (PRR) resulting in decreased type I interferon (IFN) production and viral clearance. The suppressor effect of type I IFN on macrophage function and IFN-γ activation are dampened leading to early and sustained low level IFN-γ release. This altered IFN-γ response promotes premature M1 polarization, and uncovers the suppressor effect on M2 activation, initiating an advanced and persistent stimulation of Th1 and Th2 type immune responses. Clinical complications in patients arise from the sustained innate and adaptive immune responses that amplify over time causing the cytokine storm characteristic of COVID-19.

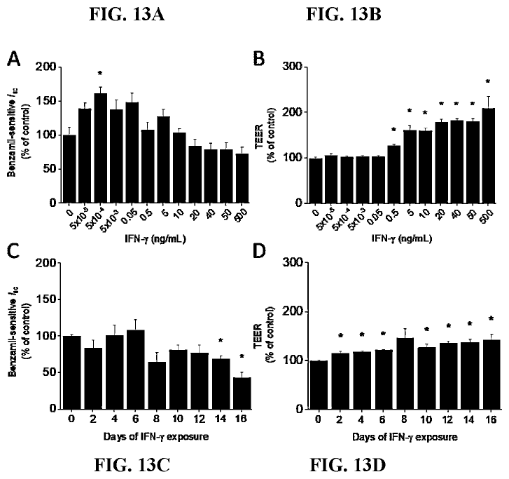

High individual variation in benzamil-sensitive current and TEER in HBECs. In an Ussing chamber-based experimental design, basal short-circuit current (I_{sc}) and transepithelial electrical resistance (TEER) were recorded in differentiated HBECs from two lung donors that were grown on snapwells at an air-liquid interface for 28 to 35 days. Benzamil, a potent ENaC blocker was used to determine ENaC activity by calculating benzamil-sensitive I_{sc} from changes in I_{sc} that occur 15 minutes after adding 6 μM benzamil to the apical side of cells. Benzamil-sensitive I_{sc} (38±2.6 μA·cm⁻², 25.7±2.2 μA·cm⁻²; P<0.01, n=10) and basal TEER (130.5±6.8 Ohm·cm², 177.7±16 Ohm·cm²; P<0.03, n=10) of age-matching HBECs differed significantly between the two donors. Therefore, normalized data were used for all subsequent experiments for statistical analyses relating to FIGS. 13-18.

IFN-γ altered ENaC activity and epithelial barrier in a dose- and time-dependent manner. IFNs play a central role during innate immune responses and are the first line of defense against viral infections. As a member of the type II IFN family, IFN-γ has potent antiviral activity and was used to determine its effect on ENaC activity and barrier function. A dose-dependent effect of IFN-γ on benzamil-sensitive I_{sc} and TEER was measured by incubating HBECs with different concentrations of IFN-γ for a period of 7 days. Interestingly, exposure to IFN-γ increased benzamil-sensitive I_{sc} to 161.62±9.7% (P<0.04) of baseline values at very low concentrations (5×10⁻⁴ ng/mL), but IFN-γ >20 ng/mL had a negative effect on benzamil-sensitive I_{sc} (FIG. 13A). IFN-γ did not affect TEER at lower concentrations, however epithelial resistance increased significantly at concentrations ≥0.5 ng/mL (FIG. 13B). These studies suggest that during early stages of innate immune response, ENaC activity and barrier function are facilitated by IFN-γ in order to maintain an appropriate homeostasis of ASL and mucosal immunity. Based on the effect of IFN-γ on TEER at 0.5 ng/mL, a concentration similar to plasma levels observed during disease conditions, all subsequent experiments were performed at 1 ng/mL to ensure adequate IFN-γ response.

The time-dependent effect of IFN-γ on ENaC activity and barrier function was studied at 1 ng/mL IFN-γ over a period of 16 days. Benzamil-sensitive I_{sc} did not change within the first 12 days of exposure but started to decrease on day 14 with the lowest ENaC activity seen on day 16 (43.7±7.0%, P<0.04; FIG. 13C). In contrast, IFN-γ improved epithelial resistance early on, and gradually increased TEER over time throughout the study period (Day 16: 142.5±12.3%, P<0.04; FIG. 13D). These results suggest that IFN-γ protects and supports ENaC activity and epithelial barrier during early stages of ARDS but may turn deleterious over time.

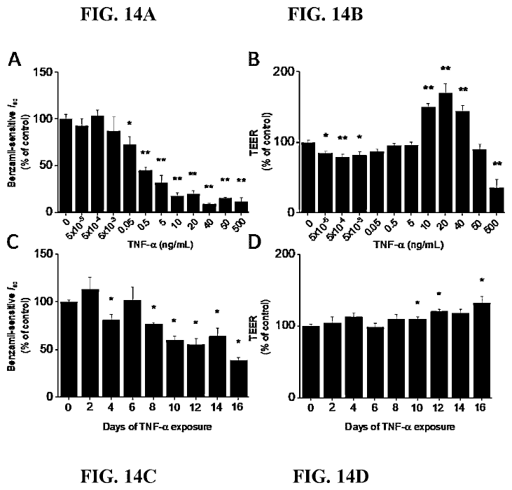

TNF-α at low concentrations disrupted ENaC function. TNF-α is one of the early and potent pro-inflammatory cytokines released during SARS-CoV-2 infection that correlates with COVID-19-associated ARDS severity. Results presented herein show that TNF-α decreased benzamil-sensitive I_{sc} at concentrations ≥0.05 ng/mL (FIG. 14A) which is similar to plasma levels seen in COVID-19 patients. Reduction in benzamil-sensitive I_{sc} plateaued at around 10 ng/mL (17.4±3.6%, P<0.01). A decrease in barrier function with increasing TNF-α concentrations was observed between 5×10⁻⁵ and 5×10⁻³ ng/mL of TNF-α (FIG. 14B). Surprisingly, between 10 and 40 ng/mL, TNF-α caused a significant increase of epithelial resistance. Because of the marked reduction in benzamil-sensitive I_{sc} at concentrations >0.5 ng/mL, TNF-α was used at 1 ng/mL for all subsequent experiments to ensure complete inhibition. When HBECs were incubated with 1 ng/mL TNF-α over a period of 16 days, benzamil-sensitive I_{sc} progressively decreased with time, starting as early as day 4 (81.2±5.4%, P<0.04), and caused a maximum reduction on day 16 (39.2±2.4%, P<0.04; FIG. 14C). No significant changes in TEER were observed within the first 8 days of exposure to TNF-α, but epithelial resistance increased with time, with peak change measured on day 16 (132.6±9.0%, P<0.04) (FIG. 14D). These studies show that TNF-α contributes significantly to disruption of ENaC activity and barrier function at concentrations associated with disease conditions, suggesting a critical role for TNF-α in the pathogenesis of ARDS.

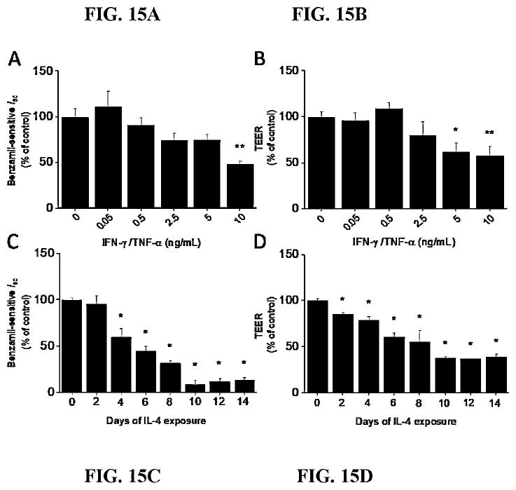

High concentrations of IFN-γ and TNF-α combination decreased ENaC and barrier function. HBECs exposed to increasing concentrations of the combination for 7 days, an experimental condition designed to mimic early stages of SARS-CoV-2 infection, resulted in a significant reduction of benzamil-sensitive I_{sc} at 10 ng/mL for each cytokine (48.0±3.7%, P<0.01) when compared to control cells. TEER decreased in the presence of the combination at 5 and 10 ng/mL (FIG. 15A, B). These results suggest that the inhibitory effect of TNF-α on ENaC function was compensated by the protective properties of IFN-γ at lower concentrations. However, the compensatory effects of IFN-γ were potentially diminished at higher concentrations, resulting in increased ENaC and barrier dysfunction, that was then driven mainly by TNF-α.

IL-4 and IL-13 caused a robust reduction in ENaC and barrier function. IL-4 and IL-13 are functionally related cytokines and initiate a Th2 immune response while repressing Th1/Th17 responses. As shown herein, the Th2 cytokines were associated with impaired ENaC function and AFC. HBECs incubated with 2 ng/mL IL-4 for 14 days significantly decreased benzamil-sensitive I_{sc} as early as day 4 (59.9±9.4%, P<0.04). Maximum reduction in benzamil-sensitive I_{sc} was seen on day 10 (8.6±5%, P<0.04), and remained suppressed for the remaining study period (FIG. 15C). Similarly, barrier function decreased as early as day 2 with maximum inhibition occurring on day 10 (37.5±2%, P<0.04) (FIG. 15D). The early and profound inhibitory effect on ENaC and epithelial barrier function in HBECs revealed that IL-4 plays a key role in the pathophysiological evolution of ARDS.

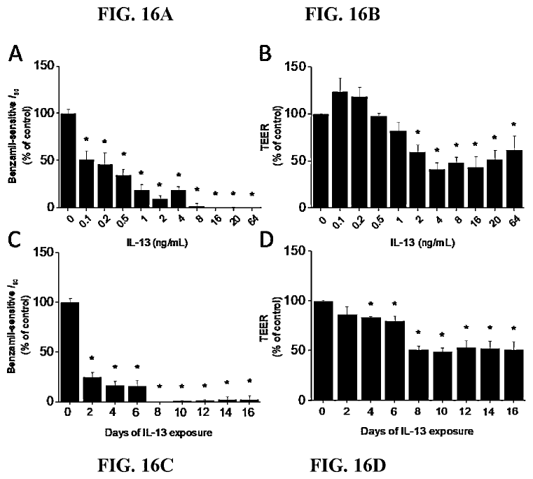

IL-4 is regulated by a positive feedback mechanism and stimulates further release of IL-4 and other Th2 cytokines (such as IL-13). Therefore, IL-13 (which lacks such properties) was used to study its contribution to disease development. When adding IL-13 to the culture medium in a dose-dependent manner, benzamil-sensitive I_{sc} progressively decreased starting at 0.1 ng/mL (50.9±9.6%, P<0.03) and benzamil-sensitive I_{sc} was completely abolished at 8 ng/mL (FIG. 16A). TEER was reduced to 59.9±7.6% (P<0.03) at 2 ng/mL IL-13, and a maximum reduction in barrier function was observed at 4 ng/mL (41.3±6.9%, P<0.03; FIG. 16B). Incubating HBECs for a period of 16 days with 20 ng/mL IL-13, decreased benzamil-sensitive I_{sc} to one-quarter of its baseline value on day 2 (25.0±5%, P<0.03) and benzamil-sensitive I_{sc} was completely suppressed by day 8 (FIG. 16C). The epithelial resistance decreased gradually over time, with a maximum reduction in TEER observed on day 10 (48.7±3.6%, P<0.03) (FIG. 16D). Together, these studies suggest an early and strong inhibitory effect of Th2-type cytokines on ENaC and barrier function, which could be responsible for an early and progressive dysregulation of ASL clearance. Since both cytokines (IL-4 and IL-13) have been detected at high concentrations in patients with COVID-19-associated ARDS, progressive impairment of AFC could lead to the onset of pulmonary edema and ARDS.

TGF-β1 decreased ENaC activity but spared barrier function. The multi-functional cytokine TGF-β1, which is generally involved in growth, proliferation and differentiation, is also part of the anti-inflammatory Treg immune response that inhibits the secretion and activation of pro-inflammatory cytokines such as IFN-γ, TNF-α, and the interleukins. Despite its immuno-suppressive nature, TGF-β1 can also act as a chemoattractant and initiate inflammation. As shown herein, TGF-β1 dysregulated ENaC trafficking and operated in sync with pro-inflammatory cytokines involved in the pathogenesis of COVID-19-associated ARDS.

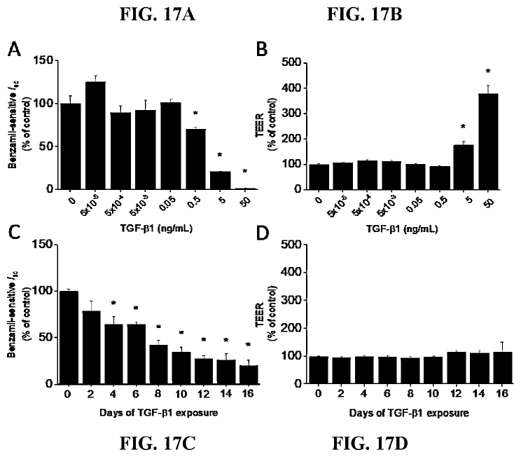

Incubating HBECs with increasing concentrations of TGF-β1 for 7 days showed that at 0.5 ng/mL, TGF-β1 reduced benzamil-sensitive I_{sc} to 70.4±2.5% (P<0.04), and at 50 ng/mL to 1.5±0.3% (P<0.04) (FIG. 17A). In contrast, TEER was not affected at low concentrations of TGF-β1 but increased gradually starting at 5 ng/mL TGF-β1 (FIG. 17B). To ensure inhibition of benzamil-sensitive I_{sc}, TGF-β1 was used at 1 ng/mL in subsequent time-dependent experiments for a maximum period of 16 days. TGF-β1 decreased benzamil-sensitive I_{sc}, starting from day 4 (64.4±8.3%, P<0.04), and benzamil-sensitive I_{sc} was reduced to 20.3±5.8% of control values by day 16 (FIG. 17C). TEER remained unaffected for the period studied (FIG. 17D). These results suggest that TGF-β1 had a dose-dependent effect on ENaC activity but had no effect on epithelial barrier function. TGF-β1 was, therefore, identified as a cytokine affecting AFC and progression into ARDS.

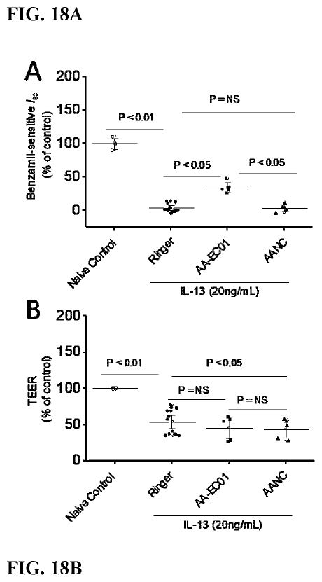

AA-EC01 improved ENaC activity abolished by high concentration of IL-13. As described herein, the present inventors developed a formulation comprising five amino acids that increased benzamil-sensitive I_{sc} (AA-EC01) and tested the formulation's ability to improve ENaC expression and function in HBECs that were incubated with IL-13 at 20 ng/mL for 14 days, a concentration and exposure time that completely abolished ENaC function. Exposure of IL-13-challenged HBECs to AA-EC01 in Ussing chambers caused an increase in benzamil-sensitive I_{sc} to 33.9±3.6% (P<0.02) when compared to 4.0±1.7% in IL-13-challenged HBECs bathed in ringer solution (FIG. 18A). When IL-13-challenged cells were exposed to a set of amino acids that were selected based on their inhibitory effect on benzamil-sensitive I_{sc} (negative control; AANC), ENaC activity remained low (3.4±2.5%, P=NS; FIG. 18A). ENaC function improved within 30 minutes after contact with AA-EC01, but was not fully restored during the study period. In contrast, IL-13-induced barrier disruption remained unchanged by AA-EC01 (FIG. 18B).

AA-EC01 restored apical ENaC expression in the presence of IL-13. Results presented herein demonstrated that the Th2 cytokines IL-4 and IL-13 were major cytokines responsible for dysregulation of ENaC activity in HBECs, and AA-EC01 improved ENaC function following cytokine incubation (FIG. 18A). Immunofluorescence imaging of HBECs showed ENaC-α subunit expression along the periciliary and apical membrane. HBECs exposed to IL-13 for 14 days showed complete translocation of ENaC protein off the periciliary and apical membrane to the sub-apical compartment and cytoplasm of ciliated and non-ciliated cells. Treatment with AA-EC01 for one hour increased immunofluorescence of ENaC-α along the apical and periciliary membrane. These observations indicate that AA-EC01 improved ENaC function at least by restoring expression of ENaC at the apical and periciliary membrane.

AA-EC01 reduced IL-6 secretion triggered by COVID-19 cytokine combination. IL-6 is a pleiotropic pro-inflammatory cytokine that is produced by a variety of cell types including epithelial cells, tissue macrophages and monocytes in response to infection and tissue injury. Initially, IL-6 is the key stimulator for acute phase proteins that attract neutrophils and other inflammatory cells to the site of inflammation. Later, IL-6 not only promotes Th2 cell differentiation resulting in expression of IL-4, but also activates a Th17 type response while disrupting the Th17/Treg balance, a prerequisite for chronic inflammation and autoimmunity. During SARS-CoV-2 infection, IL-6 together with other pro-inflammatory cytokines such as IL-1β and TNF-α are produced by bronchial epithelial cells in response to elevated Ang II. Using immunofluorescence microscopy, the present inventors demonstrated that IL-6 expression increased along the periciliary membrane of HBECs after exposure to a cytokine combination consisting of IFN-γ, TNF-α and TGF-β1 for a period of 7 days. When cytokine-incubated cells were treated with AA-EC01 for one hour, the IL-6-associated immunofluorescence signal decreased significantly at the apical membrane. Based on these studies, the beneficial effect of AA-EC01 was not limited to enhancing ENaC function, but rather also included immuno-modulatory properties on cytokines which play key roles in COVID-19 disease evolution.

AA-EC01 reduced MUC5AC secretion induced by IL-13. MUC5AC is a gel-forming, viscous mucin that is generally produced by goblet cells at epithelial surfaces. MUC5AC expression increases substantially during lung injury and inflammation resulting in progressive airway obstruction, impaired mucosal defenses and a decline in lung function. MUC5AC is a significant contributor in the pathogenesis of asthma and cystic fibrosis and is also upregulated by numerous pathogens and endogenous factors associated with inflammation. During respiratory viral infections, overexpression of MUC5AC is particularly triggered by increased production of TNF-α and Th2 type cytokines. The present inventors used immunofluorescence imaging to reveal goblet cell hyperplasia and increased expression and secretion of MUC5AC after IL-13 incubation. Treatment with AA-EC01 for one hour reduced intra- and extracellular MUC5AC in affected cells, suggesting that AA-EC01 had the potential to regulate mucus production in bronchial epithelial cells. Because critically ill patients with COVID-19 present with airway obstruction that correlated with high levels of MUC5AC in their sputum, MUC5AC may also serve as a target for AA-EC01.

In summary, extreme disparities in the way SARS-CoV-2-associated molecular patterns are recognized by PRR cause unpredictable and highly variable activation of innate and adaptive immune responses and release of associated cytokines (IFNs, Th1, Th2, Th17 and Treg). In cases of an escalated immune response, patients present with pulmonary edema or ARDS, a manifestation of the cytokine storm syndrome (FIG. 1). Results presented herein demonstrate that these cytokines impair ENaC and barrier function in airway epithelium. ENaC function is crucial for regulation of ASL and precise maintenance of a thin layer of fluid on the surface of alveolar epithelium is critical for efficient gas exchange. The barrier defect results in alveolar-capillary hyper-permeability and leakage of protein-rich fluid from pulmonary capillaries into the interstitial and alveolar space, causing decreased oxygen saturation. Currently, treatment of ARDS is mostly supportive and consists of oxygen supplementation and ventilator support. The ventilator-delivered oxygen is depleted in part by oxygenation of excess fluid within the alveoli, thereby decreasing the oxygen available for exchange across the blood-air barrier and uncoupling endothelial nitric oxide synthase (eNOS), which is associated with formation of superoxide and peroxynitrite. Peroxynitrite causes irreversible nitration of tyrosine residues in various cellular proteins, including ENaC and barrier proteins leading to collagen deposition, fibrosis and tissue remodeling as the condition progresses. Mechanical ventilation causes additional damage to the lung parenchyma resulting in ventilator-induced lung injury which could explain the high mortality (65-88%) in affected patients. Moreover, patients who survived intubation exhibited reduced lung function with significant scarring. Therefore, supportive therapy worsens lung injury and weaning patients off ventilator support becomes progressively more difficult over time. Alveolar fluid accumulation is a prominent cause of morbidity and mortality in ARDS associated with SARS-CoV-2 and other infections, but few options are available with respect to therapeutic agents that effectively target ENaC and barrier function.

As shown herein, AA-EC01 enhanced ENaC function in HBECs and therefore, is a promising therapeutic formulation for use in clinical intervention to improve AFC and to treat pulmonary edema and ARDS. AA-EC01 was shown to increase ENaC function in HBECs exposed to pathologically high concentrations of cytokines characteristic of cytokine storm syndrome for a period sufficient to abolish ENaC function. Additionally, AA-EC01 decreased the production and secretion of IL-6 and MUC5AC.

TNF-α is a potent pro-inflammatory cytokine that has pleiotropic effects with multiple homeostatic and pathologic mechanisms and its levels are elevated during ARDS. TNF-α decreased α- β- and γ-ENaC mRNA, protein levels and amiloride-sensitive I_{sc} in alveolar epithelial cells. TNF-α downregulates the expression of tight junction proteins while increasing alveolar permeability. In the present study, TNF-α at lower concentrations had no effect on benzamil-sensitive I_{sc}, while higher concentrations resulted in a significant decrease in ENaC activity. In contrast, a reduction in TEER was seen at lower concentrations while higher concentrations increased epithelial resistance.

Dysregulation of ENaC function begins with TMPRSS2 that cleaves and activates SARS-CoV-2, since ENaC has cleavage sites similar to those of the SARS-CoV-2 spike protein. ENaC function is further reduced by elevated Ang II and kinins. Inhibition of ENaC and barrier functions by various cytokines released during SARS-CoV-2 infection is primarily responsible for ARDS and persists long after the virus ceases its replication. In the present studies, prolonged incubation of HBECs with a lower concentration of IFN-γ inhibited ENaC function. The gradual decrease in benzamil-sensitive I_{sc} in HBECs when incubated with IFN-γ for ≥14 days could help explain the disease progression observed in SARS-CoV-2. Elevated plasma IFN-γ and IL-6 levels have been reported in severe COVID-19 patients when compared to those with mild disease. IFN-γ rarely acts alone, and together with TNF-α, it has been shown to upregulate inducible nitric oxide synthase (iNOS) in macrophages. This is particularly important as eNOS uncoupling triggers superoxide and peroxynitrite formation which damage proteins resulting in decreased ENaC and barrier function. These effects are exacerbated with oxygen supplementation and ventilatory support where superoxide formation is increased.

The present inventors studied the combination of IFN-γ and TNF-α on HBECs for their effect on benzamil-sensitive I_{sc} and TEER. Results presented herein demonstrate that the combination of both cytokines at 10 ng/mL worked synergistically. TNF-α reduced ENaC activity when alone, but when combined with IFN-γ, the combination of TNF-α and IFN-γ also affected barrier function. These studies showed that TNF-α caused significant damage to ENaC and barrier function during early stages of COVID-19, particularly in the presence of IFN-γ.

Treg cells activate the release of TGF-β and IL-10, maintain immunological homeostasis by suppressing CD8⁺, CD4⁺ T cells, monocytes, NK cells, and B cells during inflammatory states, and play a critical role in prevention of autoimmunity. The inhibitory effects of Treg cells are diminished during COVID-19. TGF-β1 is known to reduce amiloride-sensitive ENaC activity, ENaC mRNA and protein expression of α-subunit. TGF-β1, however, has pleiotropic effects and its function depends on affiliated cytokines and the inflammatory state. During the pathogenesis of COVID-19, the complex combination of cytokines makes it more difficult to determine the specific effect of TGF-β1 on ENaC and barrier function. In the present studies, TGF-β1 tested independently of other cytokines resulted in decreased benzamil-sensitive I_{sc} at concentrations ≥0.5 ng/mL as early as day 4, with no inhibitory effect on TEER. These effects were like those observed in response to IFN-γ and TNF-α.

SARS-CoV-2 infection can lead to an impaired innate immune response characterized by an early Th1 type activation coupled with a decreased suppressor effect on the Th2 response, which results in Th1/Th2 imbalance with predominance for the Th2 response. Early Th2 activation resulting from diminished IFN-γ production activates M2 macrophages, releases Th2 cytokines and increases arginase activity. The activation of the arginase pathway decreases NO-mediated cytotoxicity by decreasing the availability of arginine for NOS, and enhances collagen synthesis, proliferation, fibrosis and tissue remodeling. IL-4 is the primary Th2 cytokine with a positive feedback response that further augments the IL-4 response, and that of other Th2 cytokines (IL-5 and IL-13). IL-4 initiates secretion of IgE from basophils as part of an allergic response, IL-5 recruits mast cells and eosinophils, and IL-13 primarily increases mucus production from epithelial cells by activating MUC5AC. IL-4 also reduces expression of β- and γ-subunits of ENaC and IL-4 and IL-13 inhibit amiloride-sensitive I_{sc}. Results presented herein demonstrate that of all cytokines studied, Th2 cytokines had a particularly profound negative effect on benzamil-sensitive I_{sc} and TEER during early stages of COVID-19 disease progression, whereas IFN-γ and TNF-α had no effect on TEER. Thus, during COVID-19 pathogenesis the early transition to a Th2 immune response in some individuals could account for more severe pulmonary events including ARDS.

Results presented herein show that IL-13 inhibited ENaC and barrier function, while AA-EC01 increased ENaC activity and expression, thereby counteracting IL-13-mediated adverse effects. The present study further demonstrated that AA-EC01 promoted translocation of ENaC from the cytoplasm to the apical membrane, where it is functionally active. Immunohistochemistry studies described herein revealed that AA-EC01 may also increase ENaC activity via increased ENaC transcription and/or ENaC protein synthesis.

Activation of Th2 type cytokines, particularly IL-13, is also a major trigger for increased production and secretion of mucins, and MUC5AC has a key role in the pathogenesis of obstructive respiratory symptoms such as those observed in patients with severe COVID-19. The inhibitory effect of AA-EC01 on intracellular MUC5AC expression and secretion in HBECs following IL-13 exposure suggested a regulatory effect of AA-EC01 on mucus production.

IL-6, a pro-inflammatory cytokine that is secreted by resident cells within the lung also plays a central role during the cytokine storm and represents a prognostic indicator in patients with COVID-19. The ability of AA-EC01 to decrease cytokine-induced IL-6 secretion in HBECs suggested that this formulation has more extensive properties that exceed its augmentation of ENaC activity.

With no approved drugs available that can reduce excessive alveolar fluid accumulation, AA-EC01 provides a solution to an unmet and urgent clinical need. Results presented herein support the use of AA-EC01 as a therapeutic agent for treating ARDS and/or for reducing the likelihood and/or severity of pulmonary complications associated with ARDS. Because AA-EC01 consists of a functional combination of amino acids with therapeutic properties, the formulation can be used as a standalone API or as a complementary API for use in combination with other treatment options. AA-EC01 has an excellent safety profile since each of the amino acids included therein is ‘generally recognized as safe’ (GRAS) and is not expected to exhibit any side effects or to be contraindicated with respect to other APIs. Accordingly, AA-EC01 in combination with standard of care APIs, could maximize the effect of standard of care therapy, thereby decreasing the duration of oxygen supplementation and ventilatory support, minimizing long term pulmonary complications, and increasing survival of affected patients. The same reasoning applies to other related amino acid formulations described herein [such as AAF03, AAF07, and the select 5AA formulation (arginine, lysine, cysteine, asparagine, and glutamine)] that reduce excessive alveolar fluid accumulation, at least in part by increasing ENaC activity.

APIs used to treat ARDS include: lung protective ventilation (low tidal volume: 6 ml/kg; moderate positive end expiratory pressure per ARDS network guidance; plateau pressure less than 30 cm water); prone positioning; high frequency oscillatory ventilation; conservative fluid strategies; low dose corticosteroids in early stages of ARDS; extracorporeal membrane oxygenation; exogenous surfactant (shown to be particularly beneficial in pediatric populations; four types: nonionic, anionic, cationic, amphoteric); immunomodulators (e.g., interleukin-1 receptor antagonists, interferon gamma and TNF-alpha inhibitors); Favipiravir (broad-spectrum RNA polymerase inhibitor); lopinavir/ritonavir (HIV protease inhibitors); umifenovir (arbidol; inhibits viral interaction and binding with host cells via ACE2); chloroquine/hydroxychloroquine (antimalarial drugs); neuromuscular agents (NMA) can be used to improve patient-ventilator synchrony and assist mechanical ventilation in patients with severe hypoxemia; inhaled nitric oxide (NO; an endogenous vasodilator); prostanoids: including prostacyclins (arachidonic acid derivatives that cause pulmonary vasodilatation); neutrophil elastase inhibitors (e.g., Depelestat); antioxidants (e.g., glutathione and its precursor N-acetylcysteine); β2 agonists; aerosolized albuterol; anti-coagulants (nebulized heparin or intravenous heparin); cell based therapies with mesenchymal stromal cells; statins; insulin; and interferon β. In combinatorial therapeutic uses, methods, and medicaments, amino acid formulations described herein may be used in combination with at least one of the above listed therapeutic interventions which are currently used to treat subjects afflicted with ARDS.

Bronchial asthma is a paroxysmal attack of breathlessness, chest tightness, and wheezing resulting from paroxysmal narrowing of the bronchial airways. Asthma is characterized by inflammation, obstruction, and hyper-responsiveness of the airway. Pathological features of bronchial asthma include bronchoconstriction and inflammation. APIs used to treat asthma, therefore, target prevention or reversal of bronchoconstriction and/or a decrease in airway inflammation.

APIs used to treat asthma are detailed hereafter. Smooth muscles of the bronchial tree mainly contain β2 receptors, stimulation of which causes bronchodilation. APIs that are sympathomimetic (cause stimulation of β2 adrenoceptors) are useful in the treatment of bronchial asthma, especially those acting mainly on β2 receptors. Such APIs include: epinephrine, ephedrine, isoproterenol, albuterol, levalbuterol, bitolterol, metaproterenol, terbutaline, ritodrine, procaterol, isoetarine, formoterol, pirbuterol, and salmeterol. Adrenaline may be administered via injection or inhaler. Adrenaline (0.3 to 0.5 mL of 1:1000 solutions) may be administered subcutaneously for asthma, which administration can be repeated after 15 to 20 minutes. It is contraindicated in elderly subjects and those suffering from ischemic heart disease, cardiac arrhythmias, or hypertension. Albuterol can be administered orally, by injection, or by inhalation. When administered orally, it is absorbed well from gastrointestinal tract and bronchodilation occurs in about 1 hour and remains for 6 to 8 hours. When administered by inhalation it acts in about 15 minutes and remains effective for 3 to 4 hours. By subcutaneous injection, its effects manifest in 5 minutes and last for 3 to 4 hours. Methyl xanthine drugs include: theophylline, aminophylline, theobromine, caffeine, oxtriphylline, dyphylline, pentoxifylline, and acefylline. Aminophylline is prescribed to patients who develop paradoxical abdominal and diaphragmatic fatigue. Aminophylline infusion is effective in improving diaphragmatic contractility. Mast cell stabilizers include: cromolyn sodium, nedocromil Na, and ketotifen. Such anti-inflammatory drugs prevent activation of inflammatory cells, particularly mast cells, eosinophils, and epithelial cells, but have no direct bronchodilator activity. They are effective in mild persistent asthma, particularly when exercise is a precipitating factor. Cromolyn sodium is derived from an Egyptian plant called khellin. It inhibits the release of chemicals from mast cells and therefore prevents all phases of asthmatic attack. It may be administered 3 to 4 times a day. The drug in powder form can be inhaled and has been developed as 1% Intel solution which is used in the nebulized device and now is available in Intel pocket inhalers. Corticosteroids include: triamcinolone, prednisone, mometasone, methylprednisolone, hydrocortisone, fluticasone, flunisolide, dexamethasone, budesonide, and beclomethasone. Corticosteroids are effective anti-inflammatory drugs. Corticosteroids reduce inflammation resulting in control of asthma manifestations and prevention of asthma exacerbation. Cortisone inhalers give local relief in asthma with minimum side effects. Cortisones are effective in asthma and persistent, abnormal breathing. 5-lipoxygenase inhibitors (e.g., zileuton) and leukotriene D4 (LTD4)-receptor antagonists (e.g., zafirlukast and montelukast) are also routinely used for treating asthma. Leukotrienes induce asthma manifestations and airway obstruction by contracting smooth muscle cells, attracting inflammatory cells, and enhancing mucus secretion and vascular permeability. In combinatorial therapeutic uses, methods, and medicaments, amino acid formulations described herein may be used in combination with at least one of the above listed therapeutic interventions which are currently used to treat subjects afflicted with asthma.

Symptoms characteristic of allergic rhinitis include: nasal congestion, nasal itch, rhinorrhea (excessive discharge of mucus from the nose), and sneezing. Second-generation oral antihistamines and intranasal corticosteroids are the mainstay of treatment. In general, therapeutic options for allergic rhinitis target reduction of symptoms. Such therapeutic options include avoidance measures (avoidance of allergens if symptoms are associated with exposure to allergens; APIs such as oral antihistamines, intranasal corticosteroids, decongestants, leukotriene receptor antagonists, and intranasal cromones; and allergen immunotherapy. Other therapies that may be useful in some subjects include decongestants and oral corticosteroids. Occasional systemic corticosteroids and decongestants (oral and topical) are also used. Over-the-counter nasal saline spray or homemade saltwater solution may also be used to flush irritants from the nasal passages and to help thin the mucus and soothe nasal passage membranes. In combinatorial therapeutic uses, methods, and medicaments, amino acid formulations described herein may be used in combination with at least one of the above listed therapeutic interventions which are currently used to treat subjects afflicted with allergic rhinitis.

Mucolytics are APIs that thin mucus, which makes the mucus easier to eliminate from the body. Mucolytics are used to treat respiratory conditions or nasal passage conditions characterized by excessive or thickened mucus. Mucolytics can be administered orally in a tablet or syrup formulation or inhaled through a nebulizer. Some of the more common types of mucolytics include: Mucinex (guaifenesin), Carbocisteine, Pulmozyme (dornase alfa), Erdosteine, Mecysteine, Bromhexine hyperosmolar saline, and mannitol powder. In combinatorial therapeutic uses, methods, and medicaments, amino acid formulations described herein may be used in combination with at least one mucolytic such as those listed above.

As used herein, the phrase “increasing ENaC activity” may be used to refer to an increase in ENaC activity of 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%.

As used herein, the phrase “increasing ENaC activity” may be used to refer to an increase in ENaC activity of one-fold, two-fold, three-fold, four-fold, five-fold, six-fold, seven-fold, eight-fold, nine-fold, ten-fold, fifteen-fold, twenty-fold, thirty-fold, forty-fold, or fifty-fold.

As used herein, the phrase “increasing ENaC activity” may be used to refer to an increase in ENaC activity to at least partially restore ENaC activity to normal levels in a particular cell or tissue, such that ENaC activity is restored to 5%, 10%, 15%, 20%, 25%, 30%, 35%, 40%, 45%, 50%, 55%, 60%, 65%, 70%, 75%, 80%, 85%, 90%, 95%, or 100% of normal ENaC activity.

As described herein, an increase or decrease in ENaC activity may be determined by, for example, measuring benzamil/amiloride sensitive current in an Ussing chamber. Based on results presented herein, AAF01, AAF03, AAF07, the select 5AA formulation (arginine, lysine, cysteine, asparagine, and glutamine) were selected as exemplary formulations that increased ENaC activity relative to a negative control solution (established as having no effect on ENaC activity) in a model system described herein that recapitulates features of respiratory distress.

Throughout the specification and claims, the following terms take the meanings explicitly associated herein, unless the context clearly dictates otherwise. The phrases “in one embodiment,” “in an embodiment,” and “in some embodiments” as used herein do not necessarily refer to the same embodiment(s), though it may. Furthermore, the phrases “in another embodiment” and “in some other embodiments” as used herein do not necessarily refer to a different embodiment, although it may. All embodiments of the disclosure are intended to be combinable without departing from the scope or spirit of the disclosure.

As used herein, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

An “effective amount” or “effective dose” of an agent (or composition containing such agent) refers to the amount sufficient to achieve a desired biological and/or pharmacological effect, e.g., when delivered to a cell or organism according to a selected administration form, route, and/or schedule. The phrases “effective amount” and “therapeutically effective amount” are used interchangeably. As will be appreciated by those of ordinary skill in this art, the absolute amount of a particular agent or composition that is effective may vary depending on such factors as the desired biological or pharmacological endpoint, the agent to be delivered, the target tissue, etc. Those of ordinary skill in the art will further understand that an “effective amount” may be contacted with cells or administered to a subject in a single dose, or through use of multiple doses, in various embodiments. In some embodiments, an effective amount is an amount that reduces excessive fluid accumulation, at least in part by increasing ENaC activity in at least one cell. In some embodiments, an effective amount is an amount that reduces excessive fluid accumulation in a subject in need thereof, at least in part by increasing ENaC activity in the subject in need thereof. In some embodiments thereof, an effective amount is an amount that reduces excessive fluid accumulation in the lungs or nasal passages of a subject in need thereof. In some embodiments, an effective amount is an amount that reduces at least one symptom of ARDS, asthma, or allergic rhinitis.

“Treat,” “treatment”, “treating” and similar terms as used herein in the context of treating a subject refer to providing medical and/or surgical management of a subject. Treatment may include, but is not limited to, administering an agent or formulation (e.g., a pharmaceutical formulation) to a subject. The term “treatment” or any grammatical variation thereof (e.g., treat, treating, and treatment etc.), as used herein, includes but is not limited to, alleviating a symptom of a disease or condition; and/or reducing, suppressing, inhibiting, lessening, or affecting the progression, severity, and/or scope of a disease or condition.

The effect of treatment may also include reducing the likelihood of occurrence or recurrence of the disease or at least one symptom or manifestation of the disease. A therapeutic agent or formulation thereof may be administered to a subject who has a disease or is at increased risk of developing a disease relative to a member of the general population. In some embodiments, a therapeutic agent or formulation thereof is administered to a subject for maintenance purposes to reduce or eliminate at least one symptom of the disease. In some embodiments, a therapeutic agent or formulation thereof may be administered to a subject who has had a disease but no longer shows evidence of the disease. The agent or formulation thereof may be administered, e.g., to reduce the likelihood of recurrence of the disease. A therapeutic agent or formulation thereof may be administered prophylactically, i.e., before development of any symptom or manifestation of a disease.

“Prophylactic treatment” refers to providing medical and/or surgical management to a subject who has not developed a disease or does not show evidence of a disease in order, e.g., to reduce the likelihood that the disease will occur or to reduce the severity of the disease should it occur. The subject may have been identified as being at risk of developing the disease (e.g., at increased risk relative to the general population or as having a risk factor that increases the likelihood of developing the disease).

The term “amelioration” or any grammatical variation thereof (e.g., ameliorate, ameliorating, and amelioration, etc.), as used herein, includes, but is not limited to, delaying the onset, or reducing the severity of a disease or condition. Amelioration, as used herein, does not require the complete absence of symptoms.

The terms “condition,” “disease,” and “disorder” are used interchangeably.

A “subject” may be any vertebrate organism in various embodiments. A subject may be an individual to whom an agent is administered, e.g., for experimental, diagnostic, and/or therapeutic purposes or from whom a sample is obtained or on whom a procedure is performed. In some embodiments a subject is a mammal, e.g., humans; a non-human primate (e.g., apes, chimpanzees, orangutans, monkeys); or domesticated animals such as dogs, cats, rabbits, cattle, oxen, horses (including, e.g., foals), pigs, sheep, goats, llamas, mice, and rats. In some embodiments, the subject is a human. The human or other mammal may be of either sex and may be at any stage of development. In some embodiments, the human or other mammal is a baby (including pre-term babies). In some embodiments, the subject has been diagnosed with ARDS, asthma, or allergic rhinitis.

Further to the above, ENaC plays an important role during childbirth. The fluid filled alveoli in a fetus is converted to air-filled alveoli at childbirth by a huge surge in ENaC expression and function. Accordingly, exemplary formulations described herein have immediate benefit in preterm infants (infants born prematurely in advance of their due dates) or infants born with a disease or disorder characterized by developmental impairments in the respiratory system. The same reasoning applies to preterm baby animals and baby animals born with a disease or disorder characterized by developmental impairments in the respiratory system.

As used herein, the term “infant” refers to human children ranging in age from birth to one year old. As used herein, the term “baby” refers to a human child ranging in age from birth to four years old, thus encompassing newborns, infants, and toddlers.

By “negligible amount” it is meant that the amino acid present does not reduce fluid accumulation in the lungs or the nasal passages. Or, in some embodiments, even if the amino acid is present in the formulation, it is not present in an amount that would affect fluid accumulation in the lungs or the nasal passages in a subject in need thereof. In some embodiments, a negligible amount is an amount wherein the total concentration of the amino acid is less than 100 mg/l, 50 mg/l, 10 mg/l, 5 mg/l, 1 mg/l, 0.5 mg/l, 0.1 mg/1, or 0.01 mg/l. In some embodiments, a negligible amount is an amount wherein the total concentration of the amino acid is less than 100 mg/l. In some embodiments, a negligible amount is an amount wherein the total concentration of the amino acid is less than 50 mg/l. In some embodiments, a negligible amount is an amount wherein the total concentration of the amino acid is less than 10 mg/l. In some embodiments, a negligible amount is an amount wherein the total concentration of the amino acid is less than 5 mg/l. In some embodiments, a negligible amount is an amount wherein the total concentration of the amino acid is less than 1 mg/l. In some embodiments, a negligible amount is an amount wherein the total concentration of the amino acid is less than 0.5 mg/l. In some embodiments, a negligible amount is an amount wherein the total concentration of the amino acid is less than 0.1 mg/l. In some embodiments, a negligible amount is an amount wherein the total concentration of the amino acid is less than 0.01 mg/l.

The term “amino acid” encompasses all known amino acids comprising an amine (—NH₂) functional group, a carboxyl (—COOH) functional group, and a side chain (“R”) group specific to each amino acid. “Amino acids” encompasses the 21 amino acids encoded by the human genome (i.e., proteinogenic amino acids), amino acids encoded or produced by bacteria or single-celled organisms, and naturally derived amino acids. For the purposes of this disclosure, the conjugate acid form of amino acids with basic side chains (arginine, lysine, and histidine) or the conjugate base form of amino acids with acidic side chains (aspartic acid and glutamic acid) are essentially the same, unless otherwise noted. “Amino acids” also encompass derivatives and analogs thereof that retain substantially the same activity in terms of increasing ENaC activity in, for example, an Ussing chamber assay. The derivatives and analogs may be, for example, enantiomers, and include both the D- and L-forms of the amino acids. The derivatives and analogs may be derivatives of “natural” or “non-natural” amino acids (e.g., β-amino acids, homo-amino acids, proline derivatives, pyruvic acid derivatives, 3-substituted alanine derivatives, glycine derivatives, ring-substituted cysteine derivatives, ring-substituted phenylalanine derivatives, linear core amino acids, and N-methyl amino acids), for example, selenocysteine, pyrrolysine, iodocysteine, norleucine, or norvaline. The derivatives and analogs may comprise a protecting group (α-amino group, α-carboxylic acid group, or suitable R group, wherein R contains a NH₂, OH, SH, COOH or other reactive functionality). Other amino acid derivatives include, but are not limited to, those that are synthesized by, for example, acylation, methylation, glycosylation, and/or halogenation of the amino acid. These include, for example, β-methyl amino acids, C-methyl amino acids, and N-methyl amino acids. The amino acids described herein may be present as free amino acids. The term “free amino acid” refers to an amino acid that is not part of a peptide or polypeptide (e.g., is not connected to another amino acid through a peptide bond). A free amino acid is free in solution (as opposed to being linked to at least one other amino acid via, for example, a dipeptide bond), but may be associated with a salt or other component in solution.

As used herein, the term “salt” refers to any and all salts and encompasses pharmaceutically acceptable salts.

The term “carrier” may refer to any diluent, adjuvant, excipient, or vehicle with which a formulation described herein is administered. Examples of suitable pharmaceutical carriers are described in Remington's Essentials of Pharmaceuticals, 21^{st} ed., Ed. Felton, 2012, which is herein incorporated by reference.

Exemplary salts for inclusion in a formulation described herein include sodium chloride, potassium chloride, calcium chloride, magnesium chloride, or tri-sodium citrate, sodium bicarbonate, sodium gluconate phosphate buffers using mono, di or tri-sodium phosphate or any combination thereof.

Exemplary diluents include calcium carbonate, sodium carbonate, calcium phosphate, dicalcium phosphate, calcium sulfate, calcium hydrogen phosphate, cellulose, microcrystalline cellulose, kaolin, sodium chloride, and mixtures thereof.

Pharmaceutically acceptable excipients used in the manufacture of pharmaceutical formulations described herein include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, and perfuming agents may also be present in the composition.

The exact amount of an amino acid formulation or composition required to achieve an effective amount will vary from subject to subject, depending, for example, on species, age, and general condition of a subject, mode of administration, and the like. An effective amount may be included in a single dose (e.g., single oral dose) or multiple doses (e.g., multiple oral doses). In some embodiments, when multiple doses are administered to a subject or applied to a tissue or cell, any two doses of the multiple doses include different or substantially the same amounts of an amino acid composition described herein. In some embodiments, when multiple doses are administered to a subject or applied to a tissue or cell, the frequency of administering the multiple doses to the subject or applying the multiple doses to the tissue or cell is as needed, three doses a day, two doses a day, one dose a day, one dose every other day, one dose every third day, one dose every week, one dose every two weeks, one dose every three weeks, or one dose every four weeks. In some embodiments, the frequency of administering the multiple doses to the subject or applying the multiple doses to the tissue or cell is one dose per day. In some embodiments, the frequency of administering the multiple doses to the subject or applying the multiple doses to the tissue or cell is two doses per day. In some embodiments, the frequency of administering the multiple doses to the subject or applying the multiple doses to the tissue or cell is three doses per day. In some embodiments, when multiple doses are administered to a subject or applied to a tissue or cell, the duration between the first dose and last dose of the multiple doses is one-third of a day, one-half of a day, one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In some embodiments, the duration between the first dose and last dose of the multiple doses is three months, six months, or one year. In some embodiments, the duration between the first dose and last dose of the multiple doses is the lifetime of the subject, tissue, or cell.

In some embodiments, a dose (e.g., a single dose or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, between 1 g and 10 g, between 1 g and 15 g, or between 1 g and 20 g, inclusive, of an amino acid formulation described herein. In some embodiments, a dose described herein includes independently between 1 mg and 3 mg, inclusive, of an amino acid formulation described herein. In some embodiments, a dose described herein includes independently between 3 mg and 10 mg, inclusive, of an amino acid formulation described herein. In some embodiments, a dose described herein includes independently between 10 mg and 30 mg, inclusive, of an amino acid formulation described herein. In some embodiments, a dose described herein includes independently between 30 mg and 100 mg, inclusive, of an amino acid formulation described herein.

Dose ranges as described herein provide guidance for the administration of pharmaceutical formulation or compositions described herein to an adult. The amount to be administered to, for example, a baby, child, or an adolescent can be determined by a medical practitioner or person skilled in the art and may be lower or the same as that administered to an adult.

All prior patents, publications, and test methods referenced herein are incorporated by reference in their entireties.

### DETAILED DESCRIPTION OF SOME EMBODIMENTS

Each of the amino acid formulations (e.g., pharmaceutical formulations) described herein may be utilized in methods for treating ARDS, asthma, or allergic rhinitis, for use in treating ARDS, asthma, or allergic rhinitis, and/or for preparing medicaments for treating ARDS, asthma, or allergic rhinitis. ARDs is characterized by excessive alveolar fluid accumulation that impedes function of the lungs. Asthma may also exhibit features of excessive fluid accumulation that impede function of the lungs. Allergic rhinitis is characterized by excessive fluid accumulation in the nasal passages. Each of the amino acid formulations described herein may be used to reduce fluid accumulation in these conditions, which ability is conferred at least in part by the ability to increase ENaC activity in the lungs or nasal passages.

In some embodiments thereof, with respect to each of the amino acid formulations (e.g., pharmaceutical formulations) described herein, the amino acid formulation does not comprise free amino acids of phenylalanine (F), glycine (G), serine (S), or N-acetyl cysteine. In some embodiments thereof, the amino acid formulation does not comprise free amino acids of at least one of phenylalanine (F), glycine (G), serine (S), or N-acetyl cysteine, or any combination thereof.

In some embodiments, the formulation comprises, consists essentially of, or consists of free amino acids, wherein the free amino acids consist essentially of or consist of lysine (K) and arginine (R) and free amino acids of at least one of glutamine (Q), tryptophan (W), tyrosine (Y), cysteine (C), or asparagine (N), or any combination thereof. Exemplary free amino acid formulations thereof include AAF01 [lysine (K), tryptophan (W), arginine (R), tyrosine (Y), and glutamine (Q)], AAF07 [K, R, Q, Y], AAF03 [K, R, Q, W], AAF02 [K, R, W], and the select 5AA formulation [K, R, Q, C, N]. In some embodiments, such free amino acid formulations thereof include AAF01 [lysine (K), tryptophan (W), arginine (R), tyrosine (Y), and glutamine (Q)], AAF07 [K, R, Q, Y], AAF03 [K, R, Q, W], and the select 5AA formulation [K, R, Q, C, N]. In some embodiments thereof, the amino acid formulation does not comprise free amino acids of phenylalanine (F), glycine (G), serine (S), or N-acetyl cysteine. In some embodiments thereof, the amino acid formulation does not comprise free amino acids of at least one of phenylalanine (F), glycine (G), serine (S), or N-acetyl cysteine, or any combination thereof.

In some embodiments, the formulation comprises, consists essentially of, or consists of free amino acids, wherein the free amino acids consist essentially of or consist of lysine (K), arginine (R), and glutamine (Q), and free amino acids of at least one of tryptophan (W), tyrosine (Y), cysteine (C), or asparagine (N), or any combination thereof. Exemplary free amino acid formulations thereof include AAF01 [lysine (K), tryptophan (W), arginine (R), tyrosine (Y), and glutamine (Q)], AAF07 [K, R, Q, Y], AAF03 [K, R, Q, W], AAF02 [K, R, W], and the select 5AA formulation [K, R, Q, C, N]. In some embodiments, such free amino acid formulations thereof include AAF01 [lysine (K), tryptophan (W), arginine (R), tyrosine (Y), and glutamine (Q)], AAF07 [K, R, Q, Y], AAF03 [K, R, Q, W], and the select 5AA formulation [K, R, Q, C, N]. In some embodiments thereof, the amino acid formulation does not comprise free amino acids of phenylalanine (F), glycine (G), serine (S), or N-acetyl cysteine. In some embodiments thereof, the amino acid formulation does not comprise free amino acids of at least one of phenylalanine (F), glycine (G), serine (S), or N-acetyl cysteine, or any combination thereof.

In some embodiments, the formulation comprises, consists essentially of, or consists of free amino acids, wherein the free amino acids consist essentially of or consist of lysine (K), arginine (R), and glutamine (Q), and free amino acids of at least one of tryptophan (W) or tyrosine (Y), or a combination thereof or free amino acids of at least one of cysteine (C) or asparagine (N), or a combination thereof. Exemplary free amino acid formulations thereof include AAF01 [lysine (K), tryptophan (W), arginine (R), tyrosine (Y), and glutamine (Q)], AAF07 [K, R, Q, Y], AAF03 [K, R, Q, W], AAF02 [K, R, W], and the select 5AA formulation [K, R, Q, C, N]. In some embodiments, such free amino acid formulations thereof include AAF01 [lysine (K), tryptophan (W), arginine (R), tyrosine (Y), and glutamine (Q)], AAF07 [K, R, Q, Y], AAF03 [K, R, Q, W], and the select 5AA formulation [K, R, Q, C, N In some embodiments thereof, the amino acid formulation does not comprise free amino acids of phenylalanine (F), glycine (G), serine (S), or N-acetyl cysteine. In some embodiments thereof, the amino acid formulation does not comprise free amino acids of at least one of phenylalanine (F), glycine (G), serine (S), or N-acetyl cysteine, or any combination thereof.

In some embodiments, the formulation comprises, consists essentially of, or consists of free amino acids, wherein the free amino acids consist essentially of or consist of lysine (K), arginine (R), and glutamine (Q), and free amino acids of at least one of tryptophan (W) or tyrosine (Y), or a combination thereof. Exemplary free amino acid formulations thereof include AAF01 [lysine (K), tryptophan (W), arginine (R), tyrosine (Y), and glutamine (Q)], AAF07 [K, R, Q, Y], and AAF03 [K, R, Q, W]. In some embodiments thereof, the amino formulation does not comprise free amino acids of phenylalanine (F), glycine (G), or serine (S). In some embodiments thereof, the amino formulation does not comprise at least one of phenylalanine (F), glycine (G), or serine (S), or any combination thereof. In some embodiments thereof, the amino acid formulation does not comprise free amino acids of phenylalanine (F), glycine (G), serine (S), or N-acetyl cysteine. In some embodiments thereof, the amino acid formulation does not comprise free amino acids of at least one of phenylalanine (F), glycine (G), serine (S), or N-acetyl cysteine, or any combination thereof.

In some embodiments, the formulation comprises, consists essentially of, or consists of free amino acids, wherein the free amino acids consist essentially of or consist of lysine (K), arginine (R), and glutamine (Q), and free amino acids of at least one of cysteine (C) or asparagine (N), or a combination thereof. Exemplary free amino acid formulations thereof include the select 5AA formulation [K, R, Q, C, N]. In some embodiments thereof, the amino acid formulation does not comprise free amino acids of phenylalanine (F), glycine (G), serine (S), or N-acetyl cysteine. In some embodiments thereof, the amino acid formulation does not comprise free amino acids of at least one of phenylalanine (F), glycine (G), serine (S), or N-acetyl cysteine, or any combination thereof.

AAF01 is an exemplary amino acid formulation described herein. A formula for determining the number of different combinations encompassed thereby is 2ⁿ−1, wherein n equals the number of different amino acids in a select list of amino acids (e.g., 5 amino acids). The total number of different combinations of lysine, tryptophan, arginine, tyrosine, and glutamine (free amino acids of AAF01) is, therefore, 31 different combinations (2⁵−1). For the sake of simplicity, each of the select amino acids is referred to with the standard single capital letters for amino acids as follows: lysine (K), tryptophan (W), arginine (R), tyrosine (Y), and glutamine (Q). The different combinations are presented in List 2 as follows: Five AA set: K, W, R, Y, Q (AAF01). Four AA subsets: K, W, R, Y; K, W, R, Q (AAF03); K, W, Y, Q; K, R, Y, Q (AAF07); and W, R, Y, Q. Three AA subsets: K, W, R (AAF02); K, W, Y; K, W, Q; K, R, Y; K, R, Q; K, Y, Q; W, R, Y; W, R, Q; W, Y, Q; and R, Y, Q. Two AA subsets: K, W; K, R; K, Y; K, Q; W, R; W, Y; W, Q; R, Y; R, Q; and Y, Q.

The formula applies to formulations (e.g., pharmaceutical formulations) comprising the select five amino acids (K W R Y Q) in AAF01 and subsets thereof comprising two, three, or four amino acid subsets of the select five amino acids and uses thereof for treating ARDS, asthma, or allergic rhinitis in a subject in need thereof and/or for preparing medicaments for treating ARDS, asthma, or allergic rhinitis.

The above formula and reasoning are equally applied to any combination of two, three, or four amino acid subsets of the select five amino acids (K W R Y Q) described herein.

In some embodiments, the formulation comprises, consists essentially of, or consists of any two free amino acids of lysine (K), tryptophan (W), arginine (R), tyrosine (Y), and glutamine (Q). Exemplary two free amino acid subsets of the 5 amino acid formulation of AAF01 [lysine (K), tryptophan (W), arginine (R), tyrosine (Y), and glutamine (Q)] are as follows: K, W; K, R; K, Y; K, Q; W, R; W, Y; W, Q; R, Y; R, Q; and Y, Q. In some embodiments, the formulation comprises, consists essentially of, or consists of K and W. In some embodiments, the formulation comprises, consists essentially of, or consists of K and R. In some embodiments, the formulation comprises, consists essentially of, or consists of K and Y. In some embodiments, the formulation comprises, consists essentially of, or consists of K and Q. In some embodiments, the formulation comprises, consists essentially of, or consists of W and R. In some embodiments, the formulation comprises, consists essentially of, or consists of W and Y. In some embodiments, the formulation comprises, consists essentially of, or consists of W and Q. In some embodiments, the formulation comprises, consists essentially of, or consists of R and Y. In some embodiments, the formulation comprises, consists essentially of, or consists of R and Q. In some embodiments, the formulation comprises, consists essentially of, or consists of Y and Q.

In some embodiments, the formulation comprises, consists essentially of, or consists of any three free amino acids of lysine (K), tryptophan (W), arginine (R), tyrosine (Y), and glutamine (Q). Exemplary three free amino acid subsets of the 5 amino acid formulation of AAF01 [lysine (K), tryptophan (W), arginine (R), tyrosine (Y), and glutamine (Q)] are as follows: K, W, R; K, W, Y; K, W, Q; K, R, Y; K, R, Q; K, Y, Q; W, R, Y; W, R, Q; W, Y, Q; and R, Y, Q. In some embodiments, the formulation comprises, consists essentially of, or consists of K, W, and R. In some embodiments, the formulation comprises, consists essentially of, or consists of K, W, and Y. In some embodiments, the formulation comprises, consists essentially of, or consists of K, W, and Q. In some embodiments, the formulation comprises, consists essentially of, or consists of K, R, and Y. In some embodiments, the formulation comprises, consists essentially of, or consists of K, R, and Q. In some embodiments, the formulation comprises, consists essentially of, or consists of K, Y, and Q. In some embodiments, the formulation comprises, consists essentially of, or consists of W, R, and Y. In some embodiments, the formulation comprises, consists essentially of, or consists of W, R, and Q. In some embodiments, the formulation comprises, consists essentially of, or consists of W, Y, and Q. In some embodiments, the formulation comprises, consists essentially of, or consists of R, Y, and Q.

In some embodiments, the formulation comprises, consists essentially of, or consists of any four free amino acids of lysine (K), tryptophan (W), arginine (R), tyrosine (Y), and glutamine (Q). Exemplary four free amino acid subsets of the 5 amino acid formulation of AAF01 [lysine (K), tryptophan (W), arginine (R), tyrosine (Y), and glutamine (Q)] are as follows: K, W, R, Y; K, W, R, Q; K, W, Y, Q; K, R, Y, Q; and W, R, Y, Q. In some embodiments, the formulation comprises, consists essentially of, or consists of K, W, R, and Y. In some embodiments, the formulation comprises, consists essentially of, or consists of K, W, R, and Q. In some embodiments, the formulation comprises, consists essentially of, or consists of K, W, Y, and Q. In some embodiments, the formulation comprises, consists essentially of, or consists of K, R, Y, and Q. In some embodiments, the formulation comprises, consists essentially of, or consists of W, R, Y, and Q.

In some embodiments, the composition comprises, consists essentially of, or consists of free amino acids of lysine (K), tryptophan (W), arginine (R), tyrosine (Y), and glutamine (Q).

The select 5AA formulation [K, R, Q, C, N] is an exemplary amino acid formulation described herein. A formula for determining the number of different combinations encompassed thereby is 2ⁿ−1, wherein n equals the number of different amino acids in a select list of amino acids (e.g., 5 amino acids). The total number of different combinations of lysine, asparagine, arginine, cysteine, and glutamine is, therefore, 31 different combinations (2⁵−1). For the sake of simplicity, each of the select amino acids is referred to with the standard single capital letters for amino acids as follows: lysine (K), asparagine (N), arginine (R), cysteine (C), and glutamine (Q). The different combinations are presented in List 1 as follows: Five AA set: K, N, R, C, Q. In some embodiments thereof, threonine (T) may optionally be added to the five AA set of K, N, R, C, Q. In some embodiments thereof, arginine (R) may be replaced by citrulline or a combination of arginine and citrulline in the five AA set of K, N, R, C, Q. Four AA subsets: K, N, R, C; K, N, R, Q; K, N, C, Q; K, R, C, Q; and N, R, C, Q. In some embodiments thereof, threonine (T) may optionally be added to any one of the four AA subsets. In some embodiments thereof, arginine (R) when present may be replaced by citrulline or a combination of arginine and citrulline in any one of the four AA subsets. Three AA subsets: K, N, R; K, N, C; K, N, Q; K, R, C; K, R, Q; K, C, Q; N, R, C; N, R, Q; N, C, Q; and R, C, Q. In some embodiments thereof, threonine (T) may optionally be added to any one of the three AA subsets. In some embodiments thereof, arginine (R) when present may be replaced by citrulline or a combination of arginine and citrulline in any one of the three AA subsets. Two AA subsets: C, N; K, R; K, C; K, Q; N, R; N, C; N, Q; R, Q; and C, Q. In some embodiments thereof, threonine (T) may optionally be added to any one of the two AA subsets. In some embodiments thereof, arginine (R) when present may be replaced by citrulline or a combination of arginine and citrulline in any one of the two AA subsets.

The formula applies to formulations (e.g., pharmaceutical formulations) comprising the select five amino acids (K N R C Q) and subsets thereof comprising two, three, or four amino acid subsets of the select five amino acids and uses thereof treating ARDS, asthma, or allergic rhinitis and for preparing medicaments for treating ARDS, asthma, or allergic rhinitis. Such formulations (e.g., pharmaceutical formulations) comprising the select five amino acids (K N R C Q) and subsets thereof comprising two, three, or four amino acid subsets of the select five amino acids include embodiments wherein, arginine (R) when present may be replaced by citrulline or a combination of arginine and citrulline.

The above formula and reasoning are equally applied to any of the two, three, or four amino acid subsets of the select five amino acids (K N R C Q) described herein.

In some embodiments, the formulation comprises, consists essentially of, or consists of any two free amino acids of lysine (K), asparagine (N), arginine (R), cysteine (C), and glutamine (Q). Exemplary two free amino acid subsets of the 5 amino acid formulation of lysine (K), asparagine (N), arginine (R), cysteine (C), and glutamine (Q) include: K, N; K, R; K, C; K, Q; N, R; N, C; N, Q; R, Q; and C, Q. In some embodiments, the formulation comprises, consists essentially of, or consists of K and N. In some embodiments, the formulation comprises, consists essentially of, or consists of K and R. In some embodiments, the formulation comprises, consists essentially of, or consists of K and C. In some embodiments, the formulation comprises, consists essentially of, or consists of K and Q. In some embodiments, the formulation comprises, consists essentially of, or consists of N and R. In some embodiments, the formulation comprises, consists essentially of, or consists of N and C. In some embodiments, the formulation comprises, consists essentially of, or consists of N and Q. In some embodiments, the formulation comprises, consists essentially of, or consists of R and Q. In some embodiments, the formulation comprises, consists essentially of, or consists of C and Q.

In some embodiments, the formulation comprises, consists essentially of, or consists of any three free amino acids of lysine (K), asparagine (N), arginine (R), cysteine (C), and glutamine (Q). Exemplary three free amino acid subsets of the 5 amino acid formulation of lysine (K), asparagine (N), arginine (R), cysteine (C), and glutamine (Q) are as follows: K, N, R; K, N, C; K, N, Q; K, R, C; K, R, Q; K, C, Q; N, R, C; N, R, Q; N, C, Q; and R, C, Q. In some embodiments, the formulation comprises, consists essentially of, or consists of K, N, and R. In some embodiments, the formulation comprises, consists essentially of, or consists of K, N, and C. In some embodiments, the formulation comprises, consists essentially of, or consists of K, N, and Q. In some embodiments, the formulation comprises, consists essentially of, or consists of K, R, and C. In some embodiments, the formulation comprises, consists essentially of, or consists of K, R, and Q. In some embodiments, the formulation comprises, consists essentially of, or consists of K, C, and Q. In some embodiments, the formulation comprises, consists essentially of, or consists of N, R, and C. In some embodiments, the formulation comprises, consists essentially of, or consists of N, R, and Q. In some embodiments, the formulation comprises, consists essentially of, or consists of N, C, and Q. In some embodiments, the formulation comprises, consists essentially of, or consists of R, C, and Q.

In some embodiments, the formulation comprises, consists essentially of, or consists of any four free amino acids of lysine (K), asparagine (N), arginine (R), cysteine (C), and glutamine (Q). Exemplary four free amino acid subsets of the 5 amino acid formulation of lysine (K), asparagine (N), arginine (R), cysteine (C), and glutamine (Q) are as follows: K, N, R, C; K, N, R, Q; K, N, C, Q; K, R, C, Q; and N, R, C, Q. In some embodiments, the formulation comprises, consists essentially of, or consists of K, N, R, and C. In some embodiments, the formulation comprises, consists essentially of, or consists of K, N, R, and Q. In some embodiments, the formulation comprises, consists essentially of, or consists of K, N, C, and Q. In some embodiments, the formulation comprises, consists essentially of, or consists of K, R, C, and Q. In some embodiments, the formulation comprises, consists essentially of, or consists of N, R, C, and Q.

In some embodiments, the formulation comprises, consists essentially of, or consists of free amino acids of lysine (K), asparagine (N), arginine (R), cysteine (C), and glutamine (Q).

In some embodiments, the formulation comprises, consists essentially of, or consists of free amino acids of arginine (R) and lysine (K) and free amino acids of at least one of tryptophan (W), tyrosine (Y), glutamine (Q), threonine (T), or asparagine (N). The different combinations of this embodiment are presented in List 3 as follows: Seven AA set: R, K, W, Y, Q, T, N. In an embodiment thereof, the formulation comprises, consists essentially of, or consists of free amino acids of R, K, W, Y, Q, T, and N. Six AA subsets: R, K, W, Y, Q, T [AAF06]; R, K, W, Y, Q, N; R, K, W, Y, T, N; R, K, W, Q, T, N; and R, K, Y, Q, T, N. In embodiments thereof, the formulation comprises, consists essentially of, or consists of free amino acids of R, K, W, Y, Q, and T [AAF06]; R, K, W, Y, Q, and N; R, K, W, Y, T, and N; R, K, W, Q, T, and N; or R, K, Y, Q, T, and N. Five AA subsets: R, K, W, Y, Q; R, K, W, Y, T [AAF04]; R, K, W, Y, N; R, K, W, Q, T [AAF05]; R, K, W, Q, N; R, K, W, T, N; R, K, Y, Q, T; R, K, Y, Q, N; R, K, Y, T, N; and R, K, Q, T, N. In embodiments thereof, the formulation comprises, consists essentially of, or consists of free amino acids of R, K, W, Y, and Q; R, K, W, Y, and T [AAF04]; R, K, W, Y, and N; R, K, W, Q, and T [AAF05]; R, K, W, Q, and N; R, K, W, T, and N; R, K, Y, Q, and T; R, K, Y, Q, and N; R, K, Y, T, and N; or R, K, Q, T, and N. Four AA subsets: R, K, W, Y; R, K, W, Q [AAF03]; R, K, W, T; R, K, W, N; R, K, Y, Q [AAF07]; R, K, Y, T; R, K, Y, N; R, K, Q, T; R, K, Q, N; and R, K, T, N. In embodiments thereof, the formulation comprises, consists essentially of, or consists of free amino acids of R, K, W, and Y; R, K, W, and Q [AAF03]; R, K, W, and T; R, K, W, and N; R, K, Y, and Q [AAF07]; R, K, Y, and T; R, K, Y, and N; R, K, Q, and T; R, K, Q, and N; or R, K, T, and N. Three AA subsets: R, K, W [AAF02]; R, K, Y; R, K, Q; R, K, T; and R, K, N. In embodiments thereof, the formulation comprises, consists essentially of, or consists of free amino acids of R, K, and W [AAF02]; R, K, and Y; R, K, and Q; R, K, and T; or R, K, and N.

Accordingly, formulations (e.g., pharmaceutical formulations) comprising the select seven amino acids (R, K, W, Y, Q, T, N) and subsets thereof comprising two (R, K), three, four, five, and six amino acid subsets of the select seven amino acids and uses thereof for treating ARDS, asthma, or allergic rhinitis in a subject in need thereof and for preparing medicaments for treating ARDS, asthma, or allergic rhinitis are encompassed herein. The above reasoning is equally applied to any combination of two (R, K), three, four, five, or six amino acid subsets of the select seven amino acids (R, K, W, Y, Q, T, N) described herein.

In some embodiments, a formulation for use in treating ARDS, asthma, or allergic rhinitis in a subject in need thereof is presented, wherein the formulation comprises, consists essentially of, or consists of a therapeutically effective combination of free amino acids, wherein the therapeutically effective combination of free amino acids consists essentially of or consists of a therapeutically effective amount of arginine and lysine; and a therapeutically effective amount of at least one of a free amino acid of cysteine, asparagine, or glutamine, or any combination thereof, wherein the therapeutically effective combination of free amino acids is sufficient to reduce fluid accumulation in the lungs associated with ARDS or asthma or to reduce fluid accumulation in the nasal passages associated with allergic rhinitis in the subject; and optionally, a pharmaceutically acceptable carrier.

In some embodiments, a formulation for use in treating ARDS, asthma, or allergic rhinitis in a subject in need thereof is presented, wherein the formulation comprises, consists essentially of, or consists of a therapeutically effective combination of free amino acids, wherein the therapeutically effective combination of free amino acids consists essentially of or consists of a therapeutically effective amount of arginine, lysine, and glutamine; and a therapeutically effective amount of at least one of a free amino acid of cysteine or asparagine or any combination thereof, wherein the therapeutically effective combination of free amino acids is sufficient to reduce fluid accumulation in the lungs associated with ARDS or asthma or to reduce fluid accumulation in the nasal passages associated allergic rhinitis; and optionally, a pharmaceutically acceptable carrier.

In some embodiments, a formulation described herein may optionally comprise monosaccharide glucose, at least one glucose-containing disaccharide, or any combination thereof, wherein the total concentration of the monosaccharide glucose, the at least one glucose-containing disaccharide, or any combination thereof is equal to or less than 90 mM. In embodiments thereof, monosaccharide glucose, the at least one glucose-containing disaccharide, or any combination thereof is equal to or less than 85 mM; monosaccharide glucose, the at least one glucose-containing disaccharide, or any combination thereof is equal to or less than 80 mM; monosaccharide glucose, the at least one glucose-containing disaccharide, or any combination thereof is equal to or less than 75 mM; monosaccharide glucose, the at least one glucose-containing disaccharide, or any combination thereof is equal to or less than 70 mM; monosaccharide glucose, the at least one glucose-containing disaccharide, or any combination thereof is equal to or less than 65 mM; monosaccharide glucose, the at least one glucose-containing disaccharide, or any combination thereof is equal to or less than 60 mM; monosaccharide glucose, the at least one glucose-containing disaccharide, or any combination thereof is equal to or less than 55 mM; monosaccharide glucose, the at least one glucose-containing disaccharide, or any combination thereof is equal to or less than 50 mM; monosaccharide glucose, the at least one glucose-containing disaccharide, or any combination thereof is equal to or less than 45 mM; monosaccharide glucose, the at least one glucose-containing disaccharide, or any combination thereof is equal to or less than 40 mM; monosaccharide glucose, the at least one glucose-containing disaccharide, or any combination thereof is equal to or less than 35 mM; monosaccharide glucose, the at least one glucose-containing disaccharide, or any combination thereof is equal to or less than 30 mM; monosaccharide glucose, the at least one glucose-containing disaccharide, or any combination thereof is equal to or less than 25 mM; monosaccharide glucose, the at least one glucose-containing disaccharide, or any combination thereof is equal to or less than 20 mM; monosaccharide glucose, the at least one glucose-containing disaccharide, or any combination thereof is equal to or less than 15 mM; monosaccharide glucose, the at least one glucose-containing disaccharide, or any combination thereof is equal to or less than 10 mM; or monosaccharide glucose, the at least one glucose-containing disaccharide, or any combination thereof is equal to or less than 5 mM.

In embodiments thereof, monosaccharide glucose, the at least one glucose-containing disaccharide, or any combination thereof ranges from 10-90 mM; ranges from 10-85 mM; ranges from 10-80 mM; ranges from 10-75 mM; ranges from 10-70 mM; ranges from 10-65 mM; ranges from 10-60 mM; ranges from 10-55 mM; ranges from 10-50 mM; ranges from 10-45 mM; ranges from 10-40 mM; ranges from 10-35 mM; ranges from 10-30 mM; ranges from 10-25 mM; ranges from 10-20 mM; ranges from 5-90 mM; ranges from 5-85 mM; ranges from 5-80 mM; ranges from 5-75 mM; ranges from 5-70 mM; ranges from 5-65 mM; ranges from 5-60 mM; ranges from 5-55 mM; ranges from 5-50 mM; ranges from 5-45 mM; ranges from 5-40 mM; ranges from 5-35 mM; ranges from 5-30 mM; ranges from 5-25 mM; ranges from 5-20 mM; ranges from 1-90 mM; ranges from 1-85 mM; ranges from 1-80 mM; ranges from 1-75 mM; ranges from 1-70 mM; ranges from 1-65 mM; ranges from 1-60 mM; ranges from 1-55 mM; ranges from 1-50 mM; ranges from 1-45 mM; ranges from 1-40 mM; ranges from 1-35 mM; ranges from 1-30 mM; ranges from 1-25 mM; or ranges from 1-20 mM.

In some embodiments, the therapeutic composition does not contain any saccharides, including any mono-, di-, oligo-, polysaccharides, and carbohydrates. In some embodiments, the therapeutic composition does not contain glucose, and/or any di-, oligo, polysaccharides, and carbohydrates that can be hydrolyzed into glucose. In some embodiments, the composition does not contain lactose. In some embodiments, the therapeutic composition does not contain fructose and/or galactose, and/or any di-, oligo, polysaccharides, and carbohydrates that can be hydrolyzed into fructose and/or galactose.

The term “consisting essentially of” as used herein, limits the scope of the ingredients and steps to the specified materials or steps and those that do not materially affect the basic and novel characteristic(s) of the present invention, e.g., formulations and use thereof for the treatment of ARDS, asthma, or allergic rhinitis and methods for treating ARDS, asthma, or allergic rhinitis. For instance, by using “consisting essentially of” the therapeutic formulation does not contain any ingredients not expressly recited in the claims including, but not limited to, free amino acids, di-, oligo, or polypeptides or proteins; and mono-, di-, oligo-, polysaccharides, and carbohydrates that have a therapeutic effect on treatment of ARDS, asthma, or allergic rhinitis. Within the context of “consisting essentially of”, a therapeutically effective amount may be determined based on a change in ENaC activity assessed by measuring benzamil sensitive current in differentiated HBECs examined in an Ussing chamber assay, wherein an ingredient that confers an increase or decrease of up to 1%, 2%, 3%, 4%, or 5% can fall within the term “consisting essentially of”.

Formulations described herein can be prepared by any method known in the art of pharmacology. In general, such preparatory methods include bringing compounds of the formulations described herein (i.e., the free amino acids into association with a carrier or excipient, and/or one or more other accessory ingredients, and then, if necessary and/or desirable, shaping, and/or packaging the product into a desired single- or multi-dose unit.

Relative amounts of the active ingredient/s, the pharmaceutically acceptable excipient, and/or any additional ingredients in a pharmaceutical formulation described herein will vary, depending upon the identity, size, and/or condition of the subject treated and further depending upon the route by which the formulation is to be administered. The formulation may comprise between 0.1% and 100% (w/w) active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or fillers or extenders such as starches, lactose, sucrose, glucose, mannitol, and silicic acid; binders such as, for example, carboxymethylcellulose, alginates, gelatin, polyvinylpyrrolidinone, sucrose, and acacia; humectants such as glycerol; disintegrating agents such as agar, calcium carbonate, potato or tapioca starch, alginic acid, certain silicates, and sodium carbonate; solution retarding agents such as paraffin; absorption accelerators such as quaternary ammonium compounds; wetting agents such as, for example, cetyl alcohol and glycerol monostearate; absorbents such as kaolin and bentonite clay; and lubricants such as talc, calcium stearate, magnesium stearate, solid polyethylene glycols, sodium lauryl sulfate, and mixtures thereof. In the case of capsules, tablets, and pills, the dosage form may include a buffering agent.

In certain embodiments, a formulation comprising amino acids described herein may be provided in powdered form and reconstituted for administration to a subject. A pharmaceutical formulation described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such formulations are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder formulations may include a solid fine powder diluent such as sugar and are conveniently provided in a unit dose form.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan and mixtures thereof. Besides inert diluents, the oral formulations can include adjuvants such as wetting agents, emulsifying and suspending agents, sweetening, flavoring, and perfuming agents. In certain embodiments for parenteral administration, the conjugates described herein are mixed with solubilizing agents such as CREMOPHOR®, alcohols, oils, modified oils, glycols, polysorbates, cyclodextrins, polymers, and mixtures thereof.

Pharmaceutical formulations described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers. Commonly available devices for inhalation include: pressurized meter dose inhalers (pMDIs), nebulizers (e.g., compressed air/jet and ultrasonic nebulizers), and dry powder inhalers (DPIs). Jet nebulizers deliver a smaller particle size and require a prolonged treatment time relative to ultrasonic nebulizers. Medications administered through inhalation are dispersed via an aerosol spray, mist, or powder that subjects inhale into their airways.

Formulations described herein as useful for pulmonary delivery may also be used for intranasal delivery of a pharmaceutical formulation described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

Variations, modifications and alterations to embodiments of the present disclosure described above will make themselves apparent to those skilled in the art. All such variations, modifications, alterations and the like are intended to fall within the spirit and scope of the present disclosure, limited solely by the appended claims.

While several embodiments of the present disclosure have been described, it is understood that these embodiments are illustrative only, and not restrictive, and that many modifications may become apparent to those of ordinary skill in the art. For example, all dimensions discussed herein are provided as examples only, and are intended to be illustrative and not restrictive.

Any feature or element that is positively identified in this description may also be specifically excluded as a feature or element of an embodiment of the present as defined in the claims.

The disclosure described herein may be practiced in the absence of any element or elements, limitation or limitations, which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the disclosure.

### EXAMPLES

### Example 1: Model System of Lung Pathology Recapitulating ARDS:IL-13-Mediated Lung Tissue Inflammation

Materials and Methods

IL-13: abcam (#ab9577); Stock solution: 10 μg/mL water; 20 ng=2 μL Stock/mL media Media change with IL-13 every other day

Experimental design: IL-13 treatment with 20 ng/mL media for 4 and 14 days. Ussing chamber experiment in Basic Ringer (5 mM glucose in basolateral side).

In some embodiments, experimental studies called for determination of:
Baseline values (30 min)
Presence or absence of 6 μM Benzamil (on apical side) (15 min)
Presence or absence of 20 μM CFTRinh 172 (on apical and basolateral sides) (15 min)
Presence or absence 10 μM CaCCinh AO1 (on apical and basolateral sides) (10 min)
Presence or absence of 20 μM Bumetanide (on basolateral side) (15 min)

For Day 0 analysis:
IL-13 treatment: 0 ng/mL media
Ussing chamber experiment in basic ringer or amino acid (AA) formulations.
In addition, S side added 5 mM Glucose

Treatment for 4 days or 14 days analysis:
IL-13 treatment: 20 ng/mL media
Ussing chamber experiment in basic ringer or AA formulations.
In addition, S side added 5 mM Glucose

In some embodiments, the day 4 and day 14 experimental studies called for determination of:
Baseline values (30 min)
Presence or absence of 6 μM Benzamil (on mucosal side) (15 min)
Presence or absence of 20 μM Bumetanide (on serosal side) (15 min)
Presence or absence of 20 μM CFTRinh 172 (on mucosal and serosal sides) (15 min)

Results

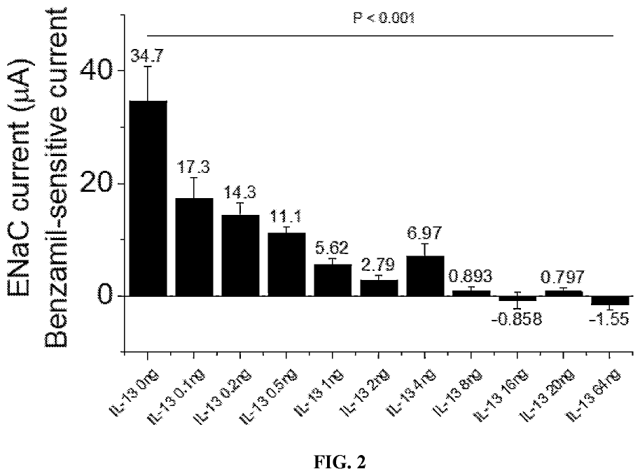

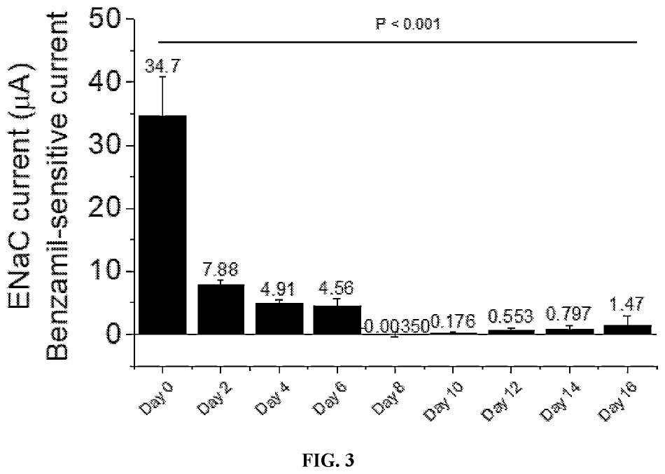

To investigate the importance of ENaC during inflammation and explore how its activity is modulated during the evolution of ARDS, the present inventors used primary cultures of human bronchial epithelial cells (HBEC) harvested from normal human lungs, which had been differentiated in vitro in an air-media interphase (air on the apical side and media on the basolateral side) for 30 days. Differentiated HBEC were used for electrophysiology experiments to evaluate the effect of IL-13 on these cells. Results from these experiments revealed an IL-13 dose-dependent reduction in ENaC current (FIG. 2). The results also showed that a maximum reduction in ENaC current occurred on day 8 of IL-13 exposure (FIG. 3). Similarly, IL-13 (20 ng/mL) caused a maximum reduction in barrier function on day 8 of exposure. These studies demonstrated that IL-13 exposure resulted in decreased ENaC activity and barrier function in differentiate HBECs. The above established that HBEC exposed to IL-13 exhibited features characteristic of lung tissue under conditions of respiratory distress and thus, provided an in vitro model system in which to evaluate efficacy of formulations for treating ARDS and asthma.

### Example 2: Testing Amino Acid Formulations Using Model System of Lung Pathology Recapitulating ARDS in Context of IL-13-Mediated in Lung Tissue Inflammation

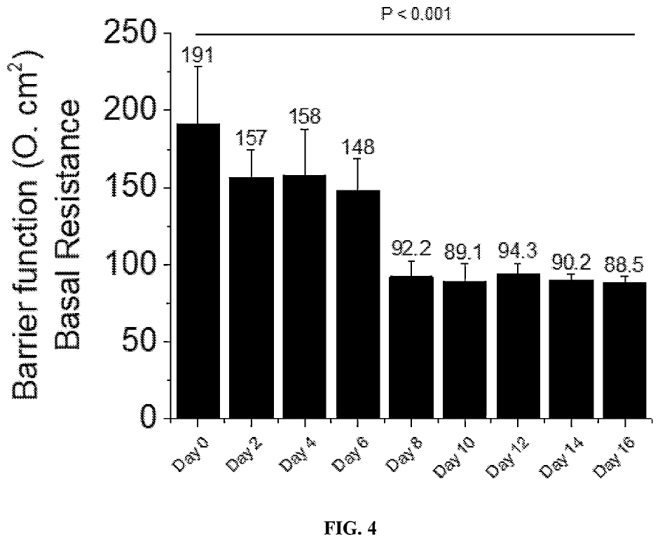

Various formulations comprising select combinations of amino acids were screened and ranked based on their ability to improve barrier function, increase electrogenic sodium absorption via ENaC (FIG. 4), and to decrease anion secretion via cystic fibrosis transmembrane conductance regulator (CFTR) and anoctamin 1 (ANO1) channels in differentiated HBEC expose to IL-13 (20 ng/mL) for 4 days or 14 days. An exemplary 5 amino acid formulation is identified (AAF01) based on these quantitative assays. Net sodium absorptive function conferred by AAF01 is validated using sodium isotope (²²Na) flux studies. AAF01 also increased electroneutral sodium absorption via sodium-hydrogen exchanger isoform 3 (NHE3). Western blot analysis showed increased protein levels of ENaC and NHE3, decreased CFTR, decreased ANO1 (a calcium-activated chloride channel), and increased levels of tight junction proteins claudin1 and E-cadherin in the presence of AAF01 in differentiated HBEC as compared to differentiated HBEC incubated in the presence of control solutions.

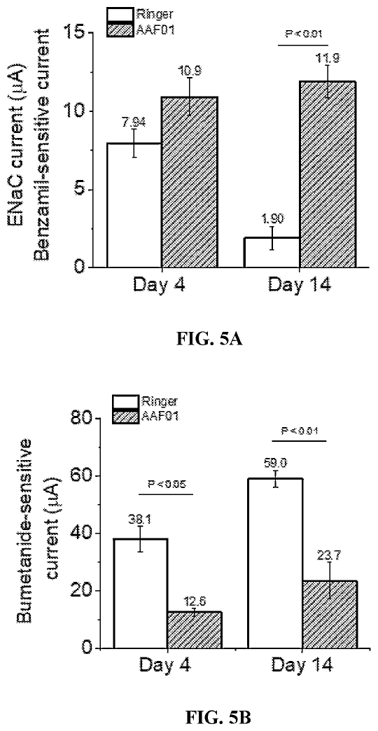

The effect of AAF01 on differentiated HBEC exposed to IL-13 for four (4) days or 14 days (FIGS. 5A and 5B) was compared to the effect of Ringers solution (negative control formulation/solution). HBEC showed increased ENaC current in the presence of the AAF01 formulation when compared to Ringer's solution at day 4 or day 14. See FIG. 5A. The AAF01-mediated increase in ENaC current was more pronounced at day 14 of IL-13 exposure, which later temporal state of the model system correlates with later stages of ARDS with respect to the pathogenesis that includes biochemical, signaling pathways engaged, integrity of the tissue and/or cells, and status of structural proteins and cell surface transport and channel proteins.

Additional experiments were performed to assess the effect of AAF01 in the presence of bumetanide, a potent inhibitor of NKCC1, which prevents chloride entry into the cell before it is available for apical exit.

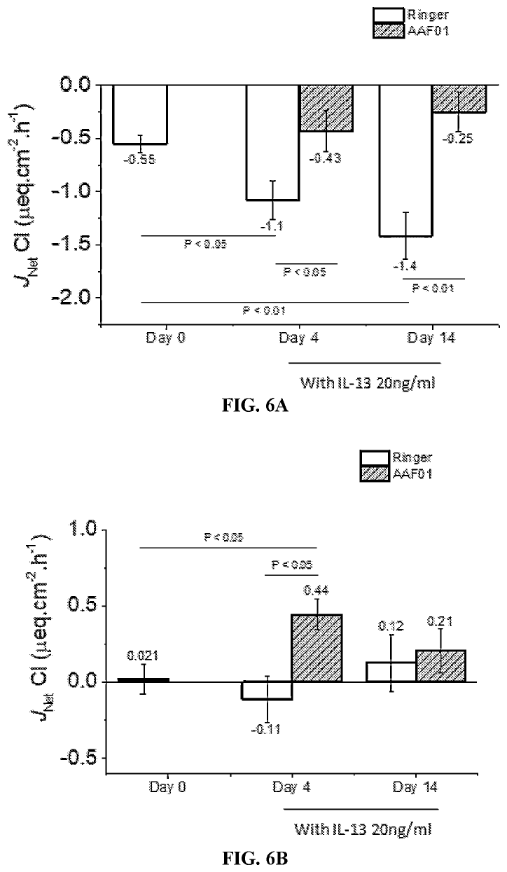

FIG. 6A presents results from isotope flux studies using ³⁶Cl showing net chloride secretion in the presence of Ringer solution (without IL-13), Ringer solution (with IL-13), or AAF01 (with IL-13) at the indicated days of incubation. AAF01 decreased chloride secretion even in the presence of IL-13. FIG. 6B presents results from isotope flux studies using ³⁶Cl showing net chloride secretion after addition of bumetanide. IL-13 increased net chloride secretion. Bumetanide-sensitive anion current is decreased in the presence of the AAF01. This decrease is not observed in the presence of Ringers solution. Accordingly, AAF01 decreases chloride secretion relative to the negative control formulation/solution used in these studies. Addition of bumetanide did not completely reverse the net chloride secretion. The presence of AAF01 did, however, result in net chloride absorption. These studies demonstrated the effectiveness of AAF01 to increase fluid uptake via enhanced ENaC activity and decreased chloride secretion, an effect that helps clear alveolar fluid as observed with ARDS or in asthma and helps clear excessive nasal secretions observed with allergic rhinitis.

Results showing increased levels of tight junction proteins claudin1 and E-cadherin in the presence of AAF01 in differentiated HBEC as compared to differentiated HBEC incubated in the presence of Ringers solution reveal that AAF01 also improved barrier function.

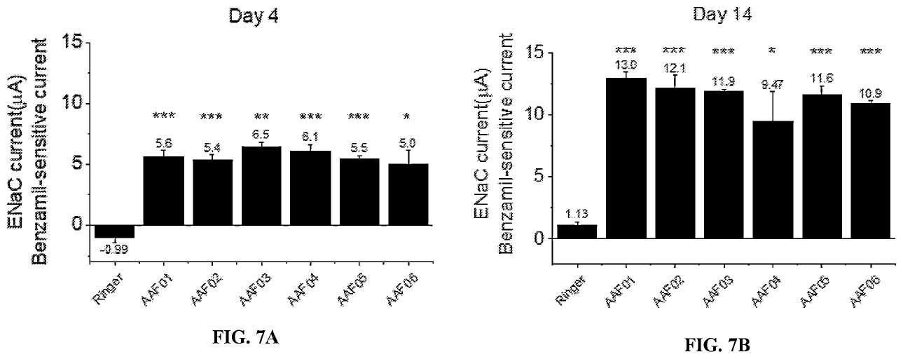

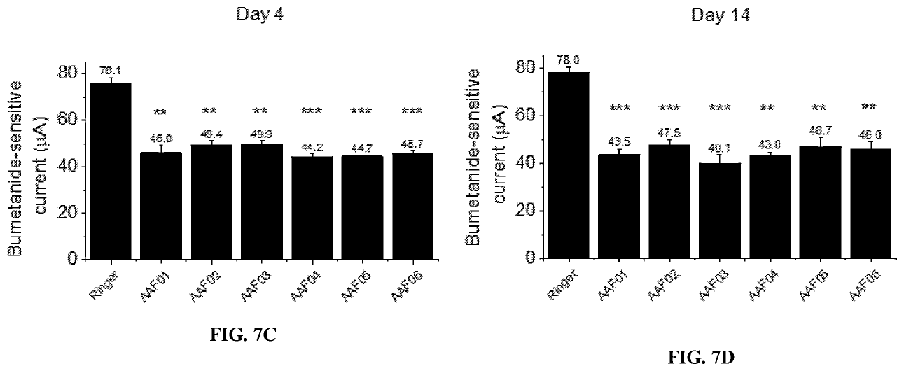

FIGS. 7A-7D present results showing that the IL-13-induced decrease in ENaC activity is significantly improved in the presence of the indicated amino acid formulations, with maximum values seen in cells exposed to AAF03 on day 4, and to AAF01 on day 14 post IL-13 treatment. The IL-13-induced increase in anion currents decreased significantly in the presence of the indicated exemplary amino acid formulations, with the lowest values observed in cells bathed in AAF04 on day 4, and in AAF03 on day 14 post IL-13 treatment.

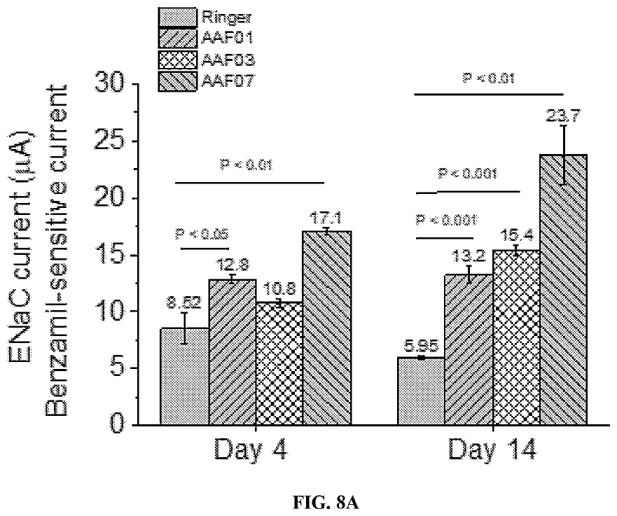

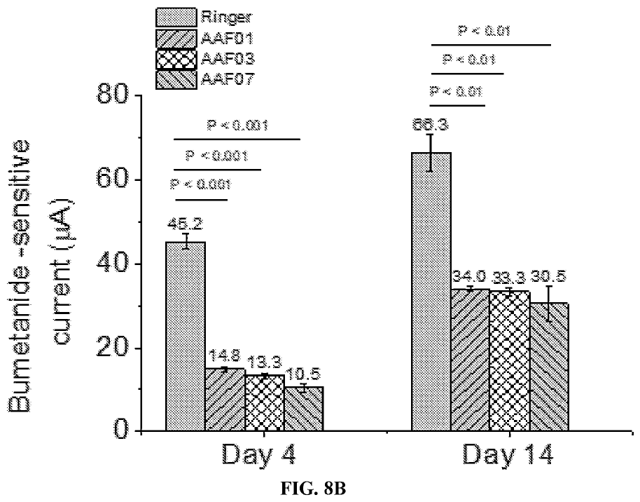

FIGS. 8A and 8B present results showing that the IL-13-induced decrease in ENaC activity is significantly improved in the presence of AAF01 or AAF07 on day 4, and AAF01, AAF03, or AAF07 on day 14 post IL-13 treatment. The IL-13-induced increase in anion current decreased significantly in HBEC exposed to the indicated exemplary amino acid formulations, with the lowest values observed in cells bathed in AAF07 on day 4 and day 14 post IL-13 treatment.

### Example 3: Model System of Lung Pathology Recapitulating ARDS: TNF-α-Mediated Lung Tissue Inflammation Using Human Bronchial Epithelial Model System

Approach: Since TNF-α has been identified as one of the major pro-inflammatory mediators implicated in the cytokine storm, the present inventors used the differentiated HBEC model system to explore the effect of amino acid formulations in the context of exposure to TNF-α as the inducer of an inflammatory state that recapitulates features of ARDS lung pathology. As described in Examples 1-2 above, amino acid formulations may be assessed for their effect on ENaC activity, anion channel activity, and barrier function in differentiated HBEC incubated in the presence of TNF-α at various concentrations and for different durations.

Methods and Materials

Ussing chamber studies may be used to determine:
Benzamil-sensitive current (Electrogenic sodium current mediated by ENaC)
Ussing chamber flux studies using ²²Na to determine net Na absorption
TEER as a measure of barrier permeability (Ohms)
Permeability assay using FITC dextran
mRNA expression of ENaC (α, β and γ), claudins 1, 2, 5, 7 and 8, occludin and E-Cadherins, acid sensing ion channels (ASIC1a) and aquaporins 1 and 5 by qRT-PCR
Western blot analysis and immunohistochemistry to determine protein levels and expression of ENaC (α, β and γ), tight junction proteins (claudins 1, 2, 5, 7 and 8, occludin and E-Cadherins), acid sensing ion channels (ASIC1a) and aquaporins 1 and 5
Determine the cytokine expression in culture media using ELISA to detect IL-6, IL-1 (3, and/or IL13.

Minimum amount of TNF-α required for maximum decrease ENaC activity and barrier function was determined by adding different concentrations of TNF-α to the culture media at, for example, 0.05, 0.1, 0.2, 0.5, 1, 2, 5, 10, 20 or 40 ng/L.

The time required for TNF-α to decrease ENaC activity and barrier function was evaluated and determined on a daily basis following its addition at, for example, 0, 1, 3, 7 or 14 days.

In some embodiments, HBECs were treated with different concentrations of TNF-α ranging from 0.00005 ng/mL to 500 ng/mL TNF-α (e.g., 0.00005, 0.0005, 0.005, 0.05, 0.5, 5, 50 or 500 ng/mL TNF-α in media) for 7 days. See FIG. 9, which shows that ENaC current decreased with increasing concentrations of TNF-α.

The AAF01 dose and time required to induce maximum increase in ENaC activity and barrier function was evaluated and determined. AAF01 was used before, together, and after TNF-α treatment. Dosing and timing of AAF01 administration was assessed in conjunction with amounts of TNF-α and duration of TNF-α exposure determined above with respect to the TNF-α-mediated lung tissue inflammation model system described herein.

Objective: To define the minimum concentration and exposure time required for AAF01 to induce a maximum increase in ENaC activity and barrier function in TNF-α treated differentiated HBECs. To achieve this, HBECs were grown on permeable snap well inserts from Costar with pores of size 0.4 μm and allowed to differentiate in an air-media interphase for a period of 30 days. Effect of TNF-α in decreasing ENaC activity, increasing CFTR and ANO1 activity, and decreasing barrier function may be evaluated as outlined below.

Determine the minimum amount of TNF-α required to induce an inflammatory effect as evidenced by a decrease in ENaC activity, an increase in CFTR and ANO1 activity, and a decrease barrier function. To achieve this, different concentrations of TNF-α may be added to the culture media, for example: 0.05, 0.1, 0.2, 0.5, 1, 2, 5, 10, 20 or 40 ng/L. The concentration of TNF-α that results in a maximal decrease in ENaC current was used in subsequent studies. These experiments were performed as described with respect to Examples 1 and 2 above.

Determine the time required for TNF-α to exert its effect as evidenced by a decrease in ENaC activity, an increase in CFTR and ANO1 activity, and a decrease barrier function. To achieve this, TNF-α was added to the media and studied on 0, 1, 3, 7 or 14 days following its addition. These studies help identify early and late responses to TNF-α and better define the progression of physiological alterations to the lung tissue following SARS-CoV-2 infection and ARDS development.

Evaluate different formulations comprising amino acids, such as those described herein (e.g., AAF01), to characterize those possessing pronounced therapeutic activity. The dose and time required for TNF-α to exert its maximum effect was determined as described above. The different formulations were assessed in parallel under different TNF-α-mediated states of inflammation correlating to different stages of lung pathology observed in ARDs progression.

Amino acid formulations were assessed for their effect on ENaC activity, anion channel activity, and barrier function in differentiated HBEC incubated in the presence of interferon-gamma (IFN-γ) alone or incubated in the presence of a combination of TNF-α and IFN-γ at various concentrations and for different durations. FIG. 10, for example, shows that ENaC current increased when cells were treated with lower concentrations of IFN-γ (0.00005 to 0.05 ng/mL media). ENaC current returned to baseline (untreated) levels when exposed to higher levels of IFN-γ, but then decreased relative to baseline when cells were treated with higher concentrations of IFN-γ (>0.05 ng/mL media). These studies help identify early and late responses to TNF-α alone, IFN-γ alone, or a combination of TNF-α and IFN-γ and better define the progression of physiological alterations to the lung tissue following SARS-CoV-2 infection and development of ARDS. The different formulations may be assessed in parallel under different TNF-α-mediated states of inflammation, IFN-γ-mediated states of inflammation, and TNF-α/IFN-γ-mediated states of inflammation correlating to different stages of lung pathology observed in ARDs progression.

The effect of TGF-β on ENaC activity in differentiated HBECs was also investigated herein. FIG. 11, for example, shows that ENaC current decreased with increasing concentrations of TGF-β1.

In summary, based on results presented herein, increasing the concentration of TNF-α revealed a concentration-dependent decrease in ENaC activity. See FIG. 9. Increasing the concentration of IFN-γ revealed an increase in activity at lower concentrations of IFN-γ and a significant decrease in ENaC activity at higher concentrations (>5 ng). See FIG. 10. Increasing the TGF-β1 concentration revealed a concentration-dependent decrease in ENaC activity. See FIG. 11.

The present inventors also evaluated ENaC activity in differentiated HBECs that were incubated in the presence of a cytokine cocktail of TNF-α, IFN-γ, and TGF-β1 for 7 days. See FIG. 12. ENaC current significantly decreased in HBECs that were exposed to the cytokine cocktail for 7 days (vehicle) relative to untreated HBECs incubated in media without the cytokine cocktail (naive). The term “vehicle” as used in FIG. 12 refers to the solution into which AAs were introduced to generate the 5AA formulation and the NC formulation and thus, serves as a negative control for the AA formulations. The select 5AA formulation (AA; arginine, lysine, cysteine, asparagine, and glutamine) conferred significant recovery of ENaC activity in HBEC exposed to TNF-α, IFN-γ, and TGF-β1 as compared to naive HBEC. In contrast, the NC formulation (aspartic acid, threonine, and leucine) did not improve the cytokine-induced reduction of ENaC activity. Indeed, the NC formulation decreased ENaC activity further in HBEC that were exposed to the cytokine cocktail relative to HBEC exposed to the cytokine cocktail and vehicle. Accordingly, in some embodiments, amino acid formulations were assessed for their ability to improve ENaC activity in the context of impaired ENaC activity such as that observed in differentiated HBECs that were incubated in the presence of a cytokine cocktail comprising TNF-α, IFN-γ, and TGF-β1 for 7 days. The results presented in FIG. 12 demonstrate the therapeutic properties of the “5AA formulation”, an exemplary formulation described herein.

Additional Materials and Methods

ENaC, IL-6 and MUC5AC expression patterns were visualized by immunofluorescence after incubation with AA-EC01 in HBECs exposed to representative cytokines. ENaC expression was assessed in naive controls and age-matched HBECs exposed to 20 ng/mL IL-13 for 14 days, that were treated with either ringer solution or AA-EC01 for one hour. IL-6 expression was assessed in naive controls and age-matched HBECs exposed to a cytokine cocktail of IFN-γ, TNF-α and TGF-β1 (1 ng/mL each) for 7 days that were treated with either ringer solution or AA-EC01 for one hour. MUC5AC expression was assessed in naive controls and age-matched HBECs exposed to 20 ng/mL IL-13 for 14 days that were treated with either ringer solution or AA-EC01 for one hour. All experiments were performed in n=2 donors on N=2 different sections. As detailed herein, AA-EC01 restored apical ENaC expression in the presence of IL-13, reduced IL-6 secretion triggered by COVID-19 cytokine combination (IFN-γ, TNF-α and TGF-β1), and reduced MUC5AC secretion induced by IL-13.

### Example 4: Model System of Lung Pathology Recapitulating ARDS: TNF-α-Mediated Lung Tissue Inflammation Using Human Alveolar Endothelial Cell Model System

Approach: To explore the effects of TNF-α on human alveolar endothelial cells, the present inventors will also use a human alveolar endothelial cell model system to explore the effect of amino acid formulations in the context of exposure to TNF-α as the inducer of an inflammatory state that recapitulates features of ARDS lung pathology. As described in Examples 1-3 above, amino acid formulations may be assessed for their effect on ENaC activity, anion channel activity, and barrier function in human alveolar endothelial cells incubated in the presence of TNF-α at various concentrations and for different durations.

Methods and Materials

Ussing chamber studies will be used to determine:
Benzamil-sensitive current (Electrogenic sodium current mediated by ENaC)
Ussing chamber flux studies using ²²Na to determine net Na absorption
TEER as a measure of barrier permeability (Ohms)
Permeability assay using FITC dextran
mRNA expression of ENaC (α, β and γ), claudins 1, 2, 5, 7 and 8, occludin and E-Cadherins, acid sensing ion channels (ASIC1a) and aquaporins 1 and 5 by qRT-PCR
Western blot analysis and immunohistochemistry to determine protein levels and expression of ENaC (α, β and γ), tight junction proteins (claudins 1, 2, 5, 7 and 8, occludin and E-Cadherins), acid sensing ion channels (ASIC1a) and aquaporins 1 and 5
Determine the cytokine expression in culture media using ELISA to detect, for example, IL-6, IL-1 β, and/or IL13.

Minimum amount of TNF-α required for maximum decrease in ENaC activity and barrier function will be determined. Different concentrations of TNF-α will be added to the culture media 0.05, 0.1, 0.2, 0.5, 1, 2, 5, 10, 20 or 40 ng/L. The time required for TNF-α to decrease ENaC activity and barrier function will be evaluated and determined. Effect of TNF-α will be studied daily following its addition at, for example, 0, 1, 3, 7 or 14 days.

The AAF01 dose and time required to induce maximum increase in ENaC activity and barrier function will be evaluated and determined. AAF01 will be used before, together, and after TNF-α treatment. Dosing and timing of administration of AAF01 to be assessed in conjunction with amounts of TNF-α and duration of TNF-α exposure determined above with respect to the TNF-α-mediated lung tissue inflammation model system described herein.

Objective: To define the minimum concentration and exposure time required for AAF01 to induce a maximum increase in ENaC activity and barrier function in TNF-α treated human alveolar endothelial cells. To achieve this, human pulmonary microvascular endothelial (HPMVE) cells may be grown on permeable snap well inserts from Costar with pores of size 0.4 μm and allowed to differentiate in media (with media on both apical and basolateral sides) for a period of 7 days. Effect of TNF-α in decreasing ENaC activity, increasing CFTR and ANO1 activity, and decreasing barrier function may be evaluated as outlined below.

Determine the minimum amount of TNF-α required to induce an inflammatory effect as evidenced by a decrease in ENaC activity, an increase in CFTR and ANO1 activity, and a decrease in barrier function. To achieve this, different concentrations of TNF-α will be added to the culture media, for example: 0.05, 0.1, 0.2, 0.5, 1, 2, 5, 10, 20 or 40 ng/L. The concentration of TNF-α that results in a maximal decrease in ENaC current will be used in subsequent studies. These experiments will be performed as described with respect to Examples 1 and 2 above.

Determine the time required for TNF-α to exert its effect as evidenced by a decrease in ENaC activity, an increase in CFTR and ANO1 activity, and a decrease barrier function. To achieve this, TNF-α will be added to the media and studied on 0, 1, 3, 7 or 14 days following its addition. These studies will help identify early and late responses to TNF-α and better define the progression of physiological alterations to the lung tissue following SARS-CoV-2 infection and development of ARDS.

Evaluate different formulations comprising amino acids, such as those described herein (e.g., AAF01), to characterize those possessing pronounced therapeutic activity. The dose and time required for TNF-α to exert its maximum effect will be determined as described above. The different formulations may be assessed in parallel under different TNF-α-mediated states of inflammation correlating to different stages of lung pathology observed in ARDs progression.

Amino acid formulations will also be assessed for their effect on ENaC activity, anion channel activity, and barrier function in human alveolar endothelial cells incubated in the presence of interferon-gamma (IFN-γ) alone or incubated in the presence of a combination of TNF-α and IFN-γ at various concentrations and for different durations. These studies will help identify early and late responses to TNF-α alone, IFN-γ alone, or a combination of TNF-α and IFN-γ and better define the progression of physiological alterations to the lung tissue following SARS-CoV-2 infection and development of ARDS. The different formulations may be assessed in parallel under different TNF-α-mediated states of inflammation, IFN-γ-mediated states of inflammation, and TNF-α/IFN-γ-mediated states of inflammation correlating to different stages of lung pathology observed in ARDs progression.

Human alveolar endothelial cells will also be tested to evaluate the effect of IL-13 on, for example, ENaC activity as per Examples 1 and 2. Exemplary amino acid formulations will be assessed for therapeutic activity with respect to human alveolar endothelial cells as indicated above with respect to HBEC.

### Example 5: Exemplary Methods Used in Examples 1-4

Electrophysiology techniques: a) Measuring benzamil-sensitive current (electrogenic sodium current mediated by ENaC), bumetanide-sensitive current and transepithelial resistance in Ussing chambers; b) Ussing chamber flux studies using ²²Na to determine net Na absorption and ³⁶Cl for chloride secretion; and c) Permeability assay using fluorescein isothiocyanate (FITC)-dextran (4 KD) added directly to the chamber.

Ussing Chamber—Sodium Flux (General)

Small intestinal mucosal tissues (ileum and jejunum) from 8-week old male Swiss mice were mounted in Ussing chambers containing isotonic Ringer solution, that was bubbled with 95% O₂ and 5% CO₂ and maintained at 37° C. throughout the experiment. After the tissues were allowed to stabilize, the conductance (G; expressed as mS/cm²) was recorded, and intestinal tissues were paired based on similar conductance. Sodium radioisotope (²²Na) was added to either the basolateral or apical side of each tissue pair (Hot). Ringer samples were taken every 15 minutes from the contralateral sides (Cold). Sample ²²Na activity was analyzed using a gamma counter, and unidirectional net sodium flux (Jnet; μeq·cm²·h⁻¹) is calculated.

$$Jnet = \frac{\begin{matrix}( Cold CPM 2 - Blank ) - \\ [ ( Cold CPM 1 - Blank ) \times 9 / 10 ] \times 5 \times 4 \times 140\end{matrix}}{( Hot CPM - Blank ) \times 10 \times 0.3}$$

[CPM=count per minute, CPM1=previous sample, CPM2=following sample; Blank=no ²²Na added; 9/10=dilution factor for each sample (0.5 mL to 5 mL); 5=chamber volume (5 mL); 4=time factor (15 min to 60 min); 140=sodium concentration; Hot CPM=“Hot” sample activity; Cold CPM=“Cold” sample activity; 10=volume factor for Hot sample (0.1 mL to 1 mL); 0.3=intestinal surface area (cm²)]

Molecular biology techniques: ENaC (α, β and γ) mRNA expression, claudins 1, 2, 5, 7 and 8, occludin and E-cadherin), acid-sensing ion channels (ASIC1a) and aquaporins 1 and 5 by qRT-PCR.

Western blot analysis and immunohistochemistry: Western blot analysis and/or immunohistochemistry to determine protein levels and expression of ENaC (α, β and γ), tight junction proteins (claudins 1, 2, 5, 7 and 8, occludin and E-cadherin), acid-sensing ion channels (ASIC1a) and aquaporins 1 and 5.

### Example 6: Improving Lung Function and Radiological Clearance in Mouse Models of Acute Respiratory Distress Syndrome (ARDS) Using AAF01

Different concentrations of exemplary formulations described herein (e.g., AAF01) may be delivered by, for example, nebulization and evaluated for therapeutic effect.

ARDS-Induction ARDS Model

Determine the time required for TNF-α to decrease ENaC activity and barrier.
Effect of TNF-α may be studied on following days after its addition 0, 1, 3, 7 or 14 days

ARDS-Induction Pneumococcus ARDS Model

Animal models of ARDS are known in the art and described in, for example, Aeffner et al. (Toxicologic Pathology, 43: 1074-1092, 2015); Gotts et al. (Am J Physiol Lung Cell Mol Physiol 317: L717-L736, 2019); and Hong et al. [Signal Transduction and Targeted Therapy (2021) 6:1], the content of each of which is incorporated herein in its entirety. Determine the AAF01 dose and time required to induce maximum increase in ENaC activity and barrier function. AAF01 will be used before, together, and after TNF-α treatment. Optimum dose and time of TNF-α identified based on information acquired in endotoxin barrier function assay and ARDS-induction ARDS model described above.

Methods
Physical measurements
Body weight, daily activity, respiratory rate, oxygen saturation, lung wet/dry weight ratio
Physiological measurements
Lung function test, permeability assay using FITC dextran (4 KD and 10 KD FITC dextran permeation studies)
Molecular biology
   mRNA expression of ENaC (α, β and γ), claudins 1, 2, 5, 7 and 8, occludin and E-Cadherins, acid sensing ion channels (ASIC 1a) and aquaporins 1 and 5 by qRT-PCR
   Western blot and immunohistochemistry analyses to determine protein levels and expression of ENaC (α, β and γ), tight junction proteins (claudins 1, 2, 5, 7 and 8, occludin and E-Cadherins), acid sensing ion channels (ASIC1a) and aquaporins 1 and 5
ELISA to determine the cytokine levels of, for example, IL-6, IL-1 β, and/or IL13.

### Example 7: Exemplary Methods Used with Respect to FIGS. 13-18

Materials and Methods

Study design. The effect of individual cytokines and combinations thereof from different stages of COVID-19 immune response (innate, Th1, Th2 and Treg) on ENaC and barrier function in HBECs was analyzed in an effort to determine their respective roles in AFC. It was hypothesized that decreased AFC is the primary trigger for pulmonary edema or ARDS as seen during COVID-19. Normal primary HBECs (P2) from two separate lung donors were used, and all experiments were performed in accordance with the guidelines and regulations described by the Declaration of Helsinki and the Huriet-Serusclat and Jardet law on human research ethics, and the protocols to obtain, culture, store and study HBECs were approved by the Institutional Review Board of the University of Florida. Age-matched differentiated HBECs were randomly divided into groups for dose- and time-dependent incubation experiments with individual cytokines and cytokine combinations, and the studies were repeated in duplicates or triplicates. Similar randomization was used when cells were treated with AA-EC01. All samples were pooled for statistical analysis. No data outliers were excluded.

HBEC cultures. HBECs were obtained from University of Alabama and University of Miami through an MTA. The cells were isolated from donor lungs as previously described (M. L. Fulcher, S. H. Randell, in Epithelial Cell Culture Protocols: Second Edition, S. H. Randell, M. L. Fulcher, Eds. (Humana Press, Totowa, N.J., 2013), pp. 109-121). Cells (P0 and P1) were plated at a concentration of 1×10⁶ cells on 10-cm, rat tail collagen I-coated cell culture dishes (ThermoFisher), and expanded in PneumaCult Ex Plus media (StemCell) containing 100 U/mL penicillin/streptomycin and 0.25 ug/mL Amphotericin B (ThermoFisher) at 37° C. and 5% CO₂/95% O₂ for 4-8 days as previously described (71). Culture medium was changed every two days until cells became 80-90% confluent.

For passaging, culture medium was removed, cells were washed with PBS, trypsinized with TrypLE Select Enzyme (ThermoFisher), and either plated on collagen I-coated cell culture dishes for further expansion (P1), or on collagen IV-coated (Sigma) permeable snapwell inserts (0.4 μM pore polycarbonate membrane, Corning) at a concentration of 80,000 cells/cm² (P2). After expansion on snapwells in PneumaCult Ex Plus containing penicillin/streptomycin to 90% confluence (cells were submerged in culture medium), cells were differentiated in PneumaCult ALI medium (StemCell) containing penicillin/streptomycin at an air-liquid interface. ALI medium was changed every two days until cells were fully differentiated (14-21 days). Differentiated HBEC are characterized by cilia motility.

Basal treatment with cytokines [IL-13 (Abcam), IL-4 (PeproTech), TNF-α, IFN-γ and TGF-β1 (R&D Systems)] diluted in ALI medium started as early as day 14 post differentiation. Individual cytokines or cytokine cocktails were added to the culture medium at the desired concentrations and cells were incubated with the cytokines for a maximum of 16 days. ALI medium containing cytokines was changed every two days. Age-matched HBECs were assigned to the following treatment groups:

I Dose-dependent studies: For 7-day treatment, IFN-γ or TNF-α were used at 5×10⁻⁵, 5×10⁻⁴, 5×10⁻³, 5×10⁻², 0.5, 5, 10, 20, 40, 50 and 500 ng/mL, while TGF-β1 was used at 5×10⁻⁵, 5×10⁻⁴, 5×10⁻³, 5×10⁻², 0.5, 5 and 50 ng/mL. For 14-day treatment, IL-13 was used at 0.1, 0.2, 0.5, 1, 2, 4, 8, 16, 20, 64 ng/mL.

II Time-dependent studies: These studies were done using a concentration that ensured maximum inhibition of benzamil-sensitive I_{sc} and TEER. HBECs were treated with respective cytokines for 2, 4, 6, 8, 10, 12, 14, or 16 days. IFN-γ, TNF-α or TGF-β1 at 1 ng/mL, IL-13 at 20 ng/mL and IL-4 at 2 ng/mL were used.

III Cytokine cocktails: were prepared using IFN-γ and TNF-α at 0.05, 0.5, 2.5, 5 and 10 ng/mL while TNF-α, IFN-γ and TGF-β1 at 1 ng/mL for each of the cytokines was added to the culture media for 7 days.

IV Treatment with amino acids for immunofluorescence: Isotonic solutions of AA-EC01, AANC (negative control) or ringer were added to the apical side of cell cultures (200 μL) that were previously incubated with either 20 ng/mL IL-13 or 1 ng/mL IFN-γ, TNF-α and TGF-β1 for 14 days or 7 days, respectively. Cell cultures were treated with the amino acids or ringer solution for one hour at 37° C. and 5% CO₂/95% O₂ before processing for immunofluorescence imaging.

Ussing chamber experiments: Snapwells with differentiated HBECs that were incubated with cytokines or age-matched HBECs without cytokine exposure were mounted in Ussing chambers (Physiologic Instruments), and cells were either bathed in isotonic ringer solution containing 113.8 mM Na⁺, 93.6 mM Cl⁻, 25 mM HCO₃⁻, 5.2 mM K⁺, 2.4 mM HPO₄⁻, 0.4 mM H₂PO₄⁻, 1.2 mM Mg²⁺, 1.2 mM Ca²⁺, and 75 mM mannitol, or in AA-EC01. Glucose (5 mM) was added to the basal side, and chambers were bubbled with 95% O₂ and 5% CO₂ at 37° C. AA-EC01 contained 8 mM lysine, 8 mM tryptophan, 8 mM arginine, 8 mM glutamine, and 1.2 mM tyrosine, and AANC contained 8 mM leucine, 8 mM cysteine, 8 mM isoleucine, 8 mM aspartic acid and 8 mM glutamate (Ajinomoto), both diluted in an electrolyte solution containing 113.8 mM Na^{t}, 93.6 mM Cl⁻, 25 mM HCO₃⁻, 5.2 mM K⁺, 2.4 mM HPO₄⁻, 0.4 mM H₂PO₄⁻, 1.2 mM Mg′, 1.2 mM Ca²⁺ and 40 mM mannitol at pH 7.4 and 300 mOsm. Cell cultures were allowed to equilibrate in the Ussing chambers for 30 minutes while continuously voltage clamped to 0 mV. Basal short circuit current (I_{sc}) and transepithelial electrical resistance (TEER) were recorded at 30-second intervals, and benzamil-sensitive I_{sc} was calculated from the difference of basal I_{sc} recorded after 30 minutes and I_{sc} measured at 15 minutes after adding 6 μM of benzamil (ThermoFisher) to the apical side.

Immunofluorescence imaging: After treatment with AA-EC01 or ringer solution, cells were fixed with 4% paraformaldehyde and embedded in paraffin. Cross-sections (4 μm) were mounted on silane-coated glass slides (FisherScientific), deparaffinized, rehydrated and heat pre-treated in retrieval buffer at pH 6.0 (Biocare Medical) per standard protocols. After blocking with 1% BSA and 10% normal goat serum, sections were incubated with mouse anti-human IL-6 monoclonal antibody (Abcam), rabbit anti-human ENaC-α polyclonal antibody (Abcepta) or mouse anti-human MUC5AC monoclonal antibody (Abcam) diluted in blocking buffer (1:100) overnight at 4° C. Goat-anti-mouse superclonal recombinant secondary antibody conjugated with AlexaFluor488 (ThermoFisher) was used for IL-6 and MUC5AC detection/visualization, and goat anti-rabbit superclonal recombinant secondary antibody conjugated with AlexaFluor647 (ThermoFisher) was used for ENaC-α detection/visualization at a concentration of 1 μg/mL incubated for one hour. Nuclei were stained with DAPI for 10 minutes, and cells were mounted in aqueous mounting medium (Abcam) before analysis. Signals were analyzed at 400× magnification using the Laser Scanning Olympus Fluoview FV1000 confocal microscope.

Statistical analysis: Results are presented as mean±standard error of mean (SEM). Analyses were performed with OriginPro 2018 software package. For each treatment group, values were tested for normal distribution using the Shapiro-Wilk normality test. Due to limited availability of donor lungs that resulted in small sample sizes and due to high variations between the donors, data were not normally distributed, and statistical analysis was performed on normalized values using non-parametric tests. The values were normalized to controls within the group, and data were pooled for comparison between groups. Kruskal-Wallis test was used for comparing the overall effect of ringer, AA-EC01 and AANC on benzamil-sensitive I_{sc} and TEER, and Mann Whitney U test was used for pairwise comparison within the group and for comparison between basal values for each cytokine at zero ng/mL or day zero with each concentration and time period studied. P<0.05 was considered significant, and NS indicates not significant.

Results Relating to FIGS. 13-18

FIG. 13 shows that prolonged incubation of HBECs with a lower concentration of IFN-γ inhibited ENaC function. ENaC inhibition was reflected in the gradual decrease in benzamil-sensitive I_{sc} in HBECs when incubated with IFN-γ for >14 days.

FIG. 14 shows that TNF-α inhibited ENaC activity but did not impair barrier function as reflected by TEER. In contrast, FIGS. 17A and 17B show that a combination of IFN-γ and TNF-α (each at 10 ng/mL) worked synergistically to reduce ENaC activity and impaired barrier function of HBECs.

FIGS. 15C and 15D show that HBECs incubated with 2 ng/mL IL-4 for 14 days exhibited significantly decreased benzamil-sensitive I_{sc} as early as day 4. Maximum reduction in benzamil-sensitive I_{sc} was seen on day 10 and benzamil-sensitive I_{sc} remained suppressed for the remaining study period (FIG. 15C). Similarly, barrier function decreased as early as day 2 with maximum inhibition occurring on day 10 (FIG. 15D).

FIG. 16 shows that adding IL-13 to the culture medium decreased benzamil-sensitive I_{sc} in a dose-dependent manner. Benzamil-sensitive I_{sc} progressively decreased starting at 0.1 ng/mL IL-13 and was completely abolished at 8 ng/mL (FIG. 16A). TEER was dramatically reduced at 2 ng/mL IL-13, with a maximum reduction in barrier function observed at 4 ng/mL (FIG. 16B). Incubating HBECs for a period of 16 days with 20 ng/mL IL-13, decreased benzamil-sensitive I_{sc} to one-quarter of its baseline value on day 2 and benzamil-sensitive I_{sc} was completely suppressed by day 8 (FIG. 16C). The epithelial resistance decreased gradually over time, with a maximum reduction in TEER observed on day 10 (FIG. 16D).

As shown in FIG. 17, TGF-β1 tested independently of other cytokines resulted in decreased benzamil-sensitive I_{sc} at concentrations >0.5 ng/mL as early as day 4 with no inhibitory effect on TEER.

FIG. 18 shows that IL-13 inhibited ENaC and barrier function, while AA-EC01 increased ENaC activity and expression, thereby counteracting IL-13-mediated adverse effects such as alveolar fluid accumulation. The present study also demonstrated that AA-EC01 promoted translocation of ENaC from the cytoplasm to the apical membrane, where it is functionally active. Immunohistochemistry studies described herein revealed that AA-EC01 may also increase ENaC activity via increased ENaC transcription and/or ENaC protein synthesis.

As shown by immunohistochemistry studies, AA-EC01 also reduced intracellular MUC5AC expression and secretion in HBECs following IL-13 exposure to a significant degree suggesting that AA-EC01 may be used to reduce mucus production. The ability of AA-EC01 to decrease cytokine-induced IL-6 secretion in HBECs (due to exposure to a cytokine combination consisting of IFN-γ, TNF-α and TGF-β1) further underscores that AA-EC01 has multiple therapeutic properties that address pulmonary complications associated with ARDS. AA-EC01 increased ENaC activity in HBECs following IL-13 exposure, significantly reduced MUC5AC expression and secretion in HBECs following IL-13 exposure, and significantly reduced the IL-6-associated immunofluorescence signal at the apical membrane of cytokine-incubated cells.

With no approved drugs available that can reduce alveolar fluid accumulation, AA-EC01 provides a solution to an unmet and urgent clinical need. Results presented herein support the use of AA-EC01 as a therapeutic agent for treating ARDS and/or for reducing the likelihood and/or severity of pulmonary complications associated with ARDS. Because AA-EC01 consists of functional amino acids with therapeutic properties, the formulation can be used as a standalone API or as complementary API for use in combination with other treatment options. AA-EC01 has an excellent safety profile since each of the amino acids included therein is ‘generally recognized as safe’ (GRAS) and is not expected to exhibit any side effects with other APIs. Accordingly, AA-EC01 in combination with standard of care APIs, could maximize the effect of standard of care therapy, thereby decreasing the duration of oxygen supplementation and ventilatory support, minimizing long term pulmonary complications, and increasing survival of affected patients.

1. A pharmaceutical formulation for use in treating acute respiratory distress syndrome (ARDS), asthma, or allergic rhinitis in a subject in need thereof, wherein the formulation comprises a therapeutically effective combination of free amino acids:
the free amino acids consisting essentially of or consisting of a therapeutically effective amount of free amino acids of arginine and lysine; and
a therapeutically effective amount of at least one of free amino acids of glutamine, tryptophan, tyrosine, cysteine, asparagine, or threonine, or any combination thereof,
wherein the therapeutically effective combination of free amino acids is formulated for delivery to the lungs for treating ARDS or asthma and the therapeutically effective combination of free amino acids is sufficient to reduce fluid accumulation in the lungs of the subject; or
wherein the therapeutically effective combination of free amino acids is formulated for delivery to the nasal passages for treating allergic rhinitis and the therapeutically effective combination of free amino acids is sufficient to reduce fluid accumulation in the nasal passages of the subject; and
optionally, at least one pharmaceutically acceptable carrier, buffer, electrolyte, adjuvant, excipient, or water, or any combination thereof.

2. (canceled)

3. (canceled)

4. (canceled)

5. The pharmaceutical formulation according to claim 1, wherein the concentration of arginine ranges from 4 mM to 10 mM; wherein the concentration of arginine ranges from 6 mM to 10 mM; wherein the concentration of arginine ranges from 7 mM to 9 mM; wherein the concentration of arginine ranges from 7.2 mM to 8.8 mM; or wherein the concentration of arginine is 8 mM.

6. The pharmaceutical formulation according to claim 1, wherein the therapeutically effective combination of free amino acids consists essentially of or consists of a therapeutically effective amount of free amino acids of arginine, lysine, tryptophan, tyrosine, and glutamine.

7. The pharmaceutical formulation according to claim 6, wherein arginine is present at a concentration ranging from 6 mM to 10 mM, lysine is present at a concentration ranging from 6 mM to 10 mM, tryptophan is present at a concentration ranging from 6 mM to 10 mM, tyrosine is present at a concentration ranging from 0.1 mM to 1.2 mM, and glutamine is present at a concentration ranging from 6 mM to 10 mM.

8. (canceled)

9. (canceled)

10. The pharmaceutical formulation according to claim 1, wherein the therapeutically effective combination of free amino acids consists essentially of or consists of a therapeutically effective amount of free amino acids of arginine, lysine, tryptophan, and glutamine.

11. (canceled)

12. (canceled)

13. (canceled)

14. The pharmaceutical formulation according to claim 1, wherein the therapeutically effective combination of free amino acids consists essentially of or consists of a therapeutically effective amount of free amino acids of arginine, lysine, tyrosine, and glutamine.

15. (canceled)

16. (canceled)

17. (canceled)

18. The pharmaceutical formulation according to claim 1, wherein the therapeutically effective combination of free amino acids consists essentially of or consists of a therapeutically effective amount of free amino acids of arginine, lysine, glutamine, cysteine, and asparagine.

19. (canceled)

20. (canceled)

21. (canceled)

22. The pharmaceutical formulation according to claim 1, wherein the therapeutically effective combination of free amino acids consists essentially of or consists of a therapeutically effective amount of free amino acids of arginine, lysine, and tryptophan.

23. The pharmaceutical formulation according to claim 1, wherein the combination of free amino acids consists essentially of or consists of a therapeutically effective amount of free amino acids of arginine, lysine, tryptophan, threonine, and tyrosine.

24. The pharmaceutical formulation according to claim 1, wherein the combination of free amino acids consists essentially of or consists of a therapeutically effective amount of free amino acids of arginine, lysine, tryptophan, threonine, and glutamine.

25. The pharmaceutical formulation according to claim 1, wherein the therapeutically effective combination of free amino acids consists essentially of or consists of a therapeutically effective amount of free amino acids of arginine, lysine, tryptophan, tyrosine, glutamine, and threonine.

26. The pharmaceutical formulation according to claim 1, further comprising at least one pharmaceutically acceptable carrier, buffer, electrolyte, adjuvant, excipient, or water, or any combination thereof.

27. The pharmaceutical formulation according to claim 1, wherein at least one of the free amino acids or each of the free amino acids comprises L-amino acids.

28. The pharmaceutical formulation according to claim 1, wherein the pharmaceutical formulation is formulated for administration by a pulmonary, inhalation, or intranasal route.

29. (canceled)

30. The pharmaceutical formulation according to claim 1, wherein the subject is a mammal.

31. (canceled)

32. (canceled)

33. (canceled)

34. The pharmaceutical formulation according to claim 1, wherein the subject is afflicted with coronavirus disease 2019 (COVID-19).

35. The pharmaceutical formulation according to claim 1, wherein reducing fluid accumulation in the lungs reduces at least one symptom associated with ARDS or asthma and wherein reducing fluid accumulation in the nasal passages reduces at least one symptom associated with allergic rhinitis.

36. (canceled)

37. (canceled)

38. A method for treating ARDS, asthma, or allergic rhinitis in a subject in need thereof, the method comprising:
administering to the subject in need thereof the pharmaceutical formulation of claim 1,
wherein the administering reduces fluid accumulation in the lung, thereby reducing at least one symptom associated with ARDS or asthma in the subject, or
the administering reduces fluid accumulation in the nasal passages of the subject, thereby reducing at least one symptom associated with allergic rhinitis in the subject.

39. The method of claim 38, wherein the pharmaceutical formulation or the medicament is administrable via at least one of a pulmonary, inhalation, or intranasal route, or any combination thereof.

40. (canceled)

41. (canceled)

42. (canceled)

43. (canceled)

44. (canceled)

45. (canceled)

46. (canceled)

47. (canceled)

48. A device comprising a pharmaceutical formulation of claim 1, wherein the device is configured to deliver the pharmaceutical formulation or the medicament to the lungs or nasal passages of the subject in need thereof.
